(19) United States
(12) Patent Application Publication
(10) Pub. No.: US 2012/0295285 A1
(43) Pub. Date: Nov. 22, 2012

(54) Ordered Assembly of Membrane Proteins During Differentiation of Erythroblasts

(75) Inventors: Xiuli An, Bayside, NY (US); Mohandas Narla, New York, NY (US); Jing Liu, Woodside, NY (US); Susanne Heck, Bronx, NY (US)

(73) Assignee: NEW YORK BLOOD CENTER, INC., New York, NY (US)

(21) Appl. No.: 13/548,761

(22) Filed: Jul. 13, 2012

Related U.S. Application Data
(63) Continuation-in-part of application No. 12/280,933, filed on Aug. 27, 2008, now Pat. No. 8,135,277.

(51) Int. Cl. C12Q 1/04; G01N 21/64; G01N 33/566; C12Q 1/06

(52) U.S. Cl. 435/7.25; 435/34; 435/39

(57) ABSTRACT

Disclosed herein are methods for the isolation, identification, and quantification of red blood cells and red blood cell precursors at different developmental stages. Also disclosed are methods for monitoring ex vivo proliferation and differentiation of red blood cells and red blood cell progenitors.

### CROSS REFERENCE TO RELATED APPLICATIONS

The present application is a continuation-in-part of U.S. patent application Ser. No. 12/280,933 filed Jun. 22, 2010, which claims the benefit under 35 USC §119(e) to U.S. Provisional Patent Application 61/219,700 filed Jun. 23, 2009, the entire contents of both of which are incorporated by reference herein.

### STATEMENT REGARDING FEDERALLY SPONSORED RESEARCH OR DEVELOPMENT

This invention was made with Government support under Grant Nos. DK26263, DK32094, and HL31579 awarded by the National Institutes of Health. The Government has certain rights in the invention.

### FIELD OF THE INVENTION

The identification and isolation of red blood cell precursors and the use of these methods and compositions is described.

### BACKGROUND OF THE INVENTION

The study of the maturation of red cells from proerythroblast to the mature functional red blood cell (RBC) has largely been limited to the study of morphologic and cellular changes. This lack of understanding regarding the maturation process has stymied efforts directed toward understanding inherited and acquired RBC diseases such as thalassemia (Cooley's anemia), diseases affecting RBC maturation (myelodysplastic syndromes [MDS]), malarial anemia, and bone marrow failure syndromes. The use of flow cytometry has helped better define each of the six stages of RBC maturation but there is much overlap and lack of clear differentiation between stages when using standard techniques.

Erythropoiesis is the process by which multipotent hematopoietic stem cells generate proliferating erythroid progenitors which subsequently undergo terminal erythroid differentiation to generate mature, non-nucleated erythrocytes. Two distinct erythroid progenitors have been functionally defined in colony assays, namely, the early-stage burst forming unit-erythroid (BFU-E) progenitor and the late-stage colony forming unit (CFU-E) progenitor. The earliest morphologically recognizable erythroblast in hematopoietic tissues is the proerythroblast, which undergoes three to four mitoses to produce reticulocytes. Morphologically distinct populations of erythroblasts are produced by each successive mitosis, beginning with proerythroblasts, and followed by basophilic, polychromatic, and orthochromatic erythroblasts. Finally, orthochromatic erythroblasts expel the nuclei to generate reticulocytes. Reticulocytes further mature into mature red blood cells, first in bone marrow and then in the circulation. This ordered differentiation process is accompanied by a decrease in cell size, enhanced chromatin condensation, progressive hemoglobinization, and marked changes in membrane organization.

During recent decades, detailed characterization of the protein composition and structural organization of the mature red cell membrane has led to insights into its function. The well-studied transmembrane proteins include band 3, glycophorin A (GPA), glycophorin C (GPC), Rh-associated glycoprotein (RhAG), Rh, CD47, Duffy, XK, Kell, CD44, Lu, and ICAM-4, all of which carry blood group antigens. A two-dimensional spectrin-based skeletal network consisting of α- and β-spectrin, short actin filiments, ankyrin, protein 4.1R, adducin, dematin, tropomyosin, tropomodulin, protein 4.2, and p55 has been shown to regulate membrane elasticity and stability. Mutations in some of these proteins result in loss of mechanical integrity and hemolytic anemia. The skeletal network is attached to the lipid bilayer through two major linkages. The first is through ankyrin, which itself forms part of a complex of band 3, GPA, RhAG, CD47, and ICAM-4, while the second involves protein 4.1R, GPC, and protein 55 with associated Duffy, XK, and Rh proteins. The loss of the ankyrin-dependent linkage, due to a mutation in ankyrin or band 3, results in loss of cohesion between the bilayer and the skeletal network, leading to membrane loss by vesiculation. This diminution in surface area reduces red cell life-span with consequent anemia. A number of additional transmembrane proteins, including CD44 and Lu, have been characterized, although their structural organization in the membrane has not been fully defined.

Some transmembrane proteins exhibit multiple functions. Band 3 serves as an anion exchanger, while Rh/RhAG are probably gas transporters, and Duffy functions as a chemokine receptor. Another group of transmembrane proteins, including Lu, CD44, ICAM-4, and integrins α4β1 and α5β1 mediate cell-cell and cell-extracellular matrix interactions. CD47 prevents premature removal from the circulation by its function as a marker of “self” on the outer surface where it binds to the inhibitory receptor SIRPα. Kell possess endothin-3 converting enzyme activity.

By contrast to our broad understanding of the structure and function of the membrane of the mature red blood cells, our knowledge of the erythroblast membrane protein composition and organization in early stages is sparse. Previous studies have given evidence for asynchronous synthesis and assembly of membrane proteins, in particular α- and β-spectrin, ankyrin and band 3 during erythroid differentiation. A number of studies revealed decreased levels of expression of adhesion molecules, such as α4β1 integrin, α5β1 integrin and Lu during terminal erythroid differentiation. Finally, the transferin receptor (CD71), which is expressed at high levels in erythroblasts at all stages of development, is absent from mature red blood cells.

### SUMMARY

Thus, disclosed herein are methods for isolation, identification and quantification of red blood cells at different developmental stages from mammals, including humans.

Disclosed herein is a method for isolating erythroid cells at different developmental stages, comprising identifying the desired erythroid cells in a source of human erythroid cells, wherein the isolated erythroid cells express a level of α4 integrin and band 3 is indicative of a developmental stage of the erythroid cells. The α4 integrin expression decreases and band 3 expression increases as differentiation of the erythroid cells progresses. In another embodiment, the method further comprises quantifying the number of erythroid cells in the developmental stage. In another embodiment, the erythroid cells are from a source selected from peripheral blood, bone marrow, cord blood, apheresis cells, and placenta.

In another embodiment, a method for identifying the erythroid cell maturation stage in an RBC disorder is provided, the method comprising determining the expression of α4 integrin and band 3 on erythroid cells from an individual having a disorder of erythropoiesis. The RBC disorder is a thalassemia, an RBC maturation disorder, and a bone marrow failure syndrome, such as a myelodysplastic syndrome Cooley's anemia. In another embodiment, the number of erythroid cells of a particular maturation stage in quantified in the RCS disorder.

Also provided herein is a method for monitoring ex vivo proliferation and differentiation of stem cells into hematopoietic precursors, erythroid cells, and mature red blood cells, the method comprising determining the expression of α4 integrin and band 3 on the cells, wherein the level of expression of α4 integrin and band 3 is indicative of a developmental stage of the erythroid cells. In one embodiment, the hematopoietic precursor is a red blood cell precursor. In another embodiment, the method further comprises quantifying the number of hematopoietic precursors, RBC precursors or mature RBCs in an RBC maturation or differentiation stage. In another embodiment, the erythroid cells are from a source selected from peripheral blood, bone marrow, cord blood, apheresis, stem cells, and placenta. In yet another embodiment, the stem cells are embryonic stem cells.

### BRIEF DESCRIPTION OF THE DRAWINGS

The following drawings form part of the present specification and are included to further demonstrate certain aspects of the examples disclosed herein. The disclosure may be better understood by reference to one or more of these drawings in combination with the detailed description of specific embodiments presented herein.

FIG. 1 depicts the differentiation of murine Friend Leukemia Virus-induced anemia (FVA) cells in vitro. FVA cells were collected at different time points as indicated and stained.

FIG. 2 depicts immunoblots of membrane proteins in various stages of murine erythroblasts. FIG. 2A. Transmembrane proteins: blots of SDS-PAGE of total membrane proteins were probed with antibodies against the indicated proteins. FIG. 2B. Kell and β1 integrin after N-glycosidase treatment: 0 h or 44 h FVA cells were either untreated (−) or treated (+) with N-glycosidase and probed with anti-Kell or anti-β1 integrin antibodies. FIG. 2C. Cytoskeleton proteins: Blots of SDS-PAGE of total membrane proteins were probed with antibodies against the indicated proteins.

FIG. 3 depicts flow cytometric analysis of transmembrane proteins of different stages of murine FVA cells. Different stages of FVA cells were stained with antibodies against molecules as indicated. The ordinate measures the number of cells displaying the fluorescent intensity given by the abscissa.

FIG. 4 depicts flow cytometric analysis of murine bone marrow cells. FIGS. 4A-4C. Bone marrow cells labeled with antibodies against TER119 and CD44, FIG. 4A: plot of CD44 versus TER119; FIG. 4B: plot of CD44 versus forward scatter (FSC) of all TER positive cells; FIG. 4C: the CD44 expression levels in the gated cell population. FIGS. 4D-4F. Bone marrow cells labeled with antibodies against TER119 and CD71

FIG. 5 depicts the isolation of different stages of murine erythroblasts by sorting using CD44, TER119 and FSC as markers. FIG. 5A depicts cytospin preparations of cells sorted from distinct regions as shown in FIGS. 4A and 4B were stained. FIG. 5B depicts the quantification of the purity of erythroblasts at different stages of maturation in various sorted populations using CD44.

FIG. 6 depicts a comparison of CD44 and CD71 expression in dual stained murine bone marrow cells. FIG. 6A depicts plot of CD44 versus FSC of all TER119 positive cells; FIG. 6B depicts CD44 expression levels in the gated cell population; FIG. 6C depicts CD71 expression levels in the identically gated cell populations; and FIG. 6D depicts a plot of CD44 versus CD71 of all TER119 positive cells.

FIG. 7 depicts the isolation of different stages of murine erythroblasts by sorting using CD71, TER119 and FSC as markers. FIG. 7A depicts cytospin preparations of cells sorted from distinct regions as shown in FIGS. 4A and 4B were stained. FIG. 7B depicts the quantification of the purity of erythroblasts at different stages of maturation in various sorted populations using CD71.

FIG. 8 depicts gating procedures for flow cytometry of murine erythroid cells. FIG. 8A depicts all events. FIG. 8B depicts live cells (7AAD⁻ cells) gated as P1. FIG. 8C depicts a cell population within the live cells of FIG. 8B which are CD45⁻CD11b⁻Gr1⁻ (P2). FIG. 8D depicts expression of TER119 in P2 cells (P3). FIG. 8E depicts a plot of CD44⁺ vs TER119⁺ cells from P3. FIG. 8F depicts a plot of CD44⁺ vs FSC cells from P3. FIG. 8G depicts Fraction I cells dated based on CD44 and TER119 expression. FIG. 8H depicts Fractions II-IV gated based on the cluster shown in FIG. 8F. FIG. 8I depicts the distinct stages of erythroblasts sorted from bone marrow cells.

FIG. 9 depicts immunoblot (FIG. 9A) and flow cytometric (FIG. 9B) analysis of D44, GLUT1 and CD71 during in vitro human erythropoiesis. FIG. 9C depicts quantitative analysis of CD44 and GLUT1 from day 7 to day 11. FIG. 9D depicts sorted erythroblasts based on expression levels of CD44 and GLUT1.

FIG. 10 depicts flow cytometric analysis of α4-integrin (FIG. 10A), CD44 (FIG. 10B), band 3 (FIG. 10C), and CD71 (FIG. 10D) during in vitro human erythropoiesis at 7, 9, 11, 13, 15, and 17 days. The ordinate measures the number of cells displaying the fluorescent intensity given by the abscissa.

FIG. 11 depicts flow cytometric analysis of different stages of cultured human erythroblasts using α4-integrin (ordinate) and band 3 (abscissa) as markers at 7 days, 9 days, 11 days, 13 days, 15 days and 17 days of culture.

FIG. 12 depicts the separation of distinct stages of erythroblasts using α4-integrin and band 3 as markers. FIG. 12A depicts flow cytometric analysis of α4-integrin and band 3 expression in erythroblasts at days 7 and 15 of culture. FIG. 12B depicts the morphological stages of specific populations of erythroblasts.

FIG. 13 depicts the mitotic activity of purified human erythroblasts at different states as expressed by cell number (FIG. 13A) and number of cell divisions (FIG. 13B).

### DETAILED DESCRIPTION OF THE INVENTION

Erythropoiesis is the process by which nucleated erythroid progenitors proliferate and differentiate to generate millions of non-nucleated red cells with their unique discoid shape and membrane material properties. The time-course of appearance of individual membrane proteins components during murine erythropoiesis throws new light on the understanding of the evolution of the unique features of the red cell membrane. Accumulation of all the major transmembrane and skeletal proteins of the mature red blood cell, except only actin, accrues progressively during terminal erythroid differentiation. At the same time, and in marked contrast, accumulation of various adhesion molecules decreases. In particular, the adhesion molecules CD44 (mouse) and α4 integrin (human) exhibits a progressive and dramatic decrease from proerythroblast to reticulocyte; this enabled the development of a system for distinguishing unambiguously between erythroblasts at successive developmental stages. These findings provide new insights into the genesis of red cell membrane function during erythroblast differentiation, and also offer a means of defining stage-specific defects in erythroid maturation in inherited and acquired red cell disorders and in bone marrow failure syndromes.

Embodiments of the present specification disclose, in part, a method of identifying the stage of maturation of erythroid cells in a sample. In one embodiment, the method disclosed herein comprises analyzing a sample comprising a population of erythroid cells to detect a level of cellular expression of two biomarkers, α4 integrin and band 3, wherein detection of a level of α4 integrin and band 3 identifies the stage of maturation of erythroid cells in the sample.

Also disclosed are methods of obtaining a substantially pure population of red blood cells or red blood cell precursors comprising obtaining a sample comprising a population of erythroid cells and detecting a level of cellular expression of two biomarkers, α4 integrin and band 3 which identifies the desired population of red blood cells or red blood cell precursors and obtaining a substantially pure population of the desired cells from the sample.

In yet another embodiment, the methods disclosed herein are suitable for monitoring ex vivo proliferation and differentiation of erythroid lineage stem cells. In another embodiment, the methods used herein are suitable for monitoring in vivo proliferation and differentiation of erythroid lineage stem cells.

In other embodiments, the methods disclosed herein are suitable for determining the differentiation stage of red blood cells in vivo or in vitro and in normal or disease states.

In additional embodiments, the methods disclosed herein are useful for isolating appropriate RBC precursors for producing RBCs suitable for transfusion.

As used herein, the term “erythroid” refers to any hematopoietic cells which has the potential to develop into an erythrocyte and includes mature red blood cells (RBC), RBC precursors such as nucleated RBCS, and stem cells. As used herein, the term “red blood cell” can refer to erythrocytes, erythroblasts and reticulocytes. The present inventors have surprisingly determined that the cell surface marker α4 integrin combined with band 3 can be used to determine the stage of differentiation or maturation of human red blood cells.

During terminal erythroid differentiation, proerythroblasts, the earliest morphologically recognizable nucleated erythroid cells in hematopoietic tissues, undergo three to four mitoses to generate, in humans, 2 million non-nucleated reticulocytes every second. Extensive remodeling of reticulocytes, first in the bone marrow and then in circulation results in the generation of mature circulating red cells with their unique discoid shape and membrane material properties. Accumulation of all major transmembrane and all skeletal proteins of the mature red blood cell, except actin, increase progressively during differentiation from the proerythroblast to the orthochromatic erythroblast stage. In marked contrast, accumulation of a series of adhesion molecules is highest in proerythroblasts and decreases during erythroid differentiation with very low level expression in orthochromatic erythroblasts. Disclosed herein are methods which allow separation of the successive stages in erythroid differentiation with greater reliability and precision than has been previously possible.

A number of earlier studies investigated the synthesis and assembly of membrane proteins during erythropoiesis encompassing a limited number of the major membrane proteins including spectrin, ankyrin, 4.1R, and band 3. In chicken erythroblasts transformed with avian erythroblastosis virus or S13 virus, expression of band 3 occurs later than that of the peripheral proteins, spectrin, ankyrin, and 4.1R. Similar results on accumulation of spectrin, ankyrin, 4.1R, and band 3 were also found to hold for murine and human erythropoiesis. In studies of the order of appearance of proteins that encode human blood group antigens in an in vitro culture system, Kell antigen was the first protein to be expressed.

Disclosed herein are protein expression profiles of highly synchronous erythroblast populations defining the stage-specific expression patterns of a range of proteins of the erythrocyte membrane. The accumulation of proteins involved in linking the lipid bilayer to the skeletal protein network (band 3, RhAG, ankyrin, and 4.1R) follows behind that of the components of the membrane skeleton (α- and β-spectrin, adducin and tropomodulin). Therefore, the assembly of a fully functional spectrin-based network, which determines the material properties of the membrane, is a late event in erythropoiesis. In this context, it is interesting to note that the components of the spectrin-based network, α- and β-spectrin, adducin, and tropomodulin are synthesized earlier than the linking proteins, starting at the proerythroblast stage and progressively increasing at later stages of differentiation. An exception to the general pattern is actin, another principal component of the membrane skeleton, the expression of which is highest in proerythroblasts and falls off as terminal erythroid differentiation proceeds. The implication is that actin has additional function in erythroblasts, which it probably exercises in its filamentous state in the cytoplasm, whereas only a small proportion is required to form the short protofilaments of the skeletal lattice.

Erythropoiesis in vivo occurs entirely in erythroid niches, termed “erythroblastic islands”, which are made up of a central macrophage surrounded by developing erythroblasts. Adhesive interactions in this specialized structure between the central macrophage and erythroblasts, as well as between erythroblasts and extracellular matrix proteins, play a critical role in regulating terminal erythroid differentiation. A number of proteins expressed on erythroblasts, including β1 integrin, CD44, Lu, α4 integrin, and ICAM-4, are responsible for various adhesive interactions. Five splice variants of β1 integrin, arising from alternative splicing of the cytoplasmic domain designated, β1A, β1B, β1C-1, β1C-2, and β1D, have previously been identified in various cells and two of the five known isoforms are expressed during erythroid differentiation. The discovery that the adhesion molecules are most strongly expressed in proerythoblasts and are either expressed at very low levels or not at all in orthochromatic erythroblasts indicates that adhesive interactions are dynamically regulated during terminal erythroid differentiation.

The red blood cell membrane undergoes dramatic remodeling during erythropoiesis. However, the molecular changes during this process remain largely unknown. Twenty-four proteins in various erythroblast developmental stages were derived from Friend leukemia virus (FVA)-induced anemic spleen. Except for actin, which decreases during erythropoiesis, all cytoskeleton proteins were increased. The major red cell transmembrane proteins band 3, GPA, GPC, Rh, RhAG, CD47, and Duffy were only weakly expressed in proerythroblasts but were significantly increased upon differentiation. In contrast, adhesion molecules such as CD44, β1 integrin, α4 integrin, Lu, and ICAM-4 were highly expressed in proerythroblasts but were lost or significantly decreased in late stage erythroblasts.

Notable, in mice the decrease in CD44 surface expression was in a progressive manner and coincided with the size change of erythroblasts. Analysis of murine bone marrow cells by flow cytometry using CD44 in conjunction with TER119 and forward scatter revealed six distinct populations which correspond to proerythroblasts, basophilic erythroblasts, polychromatic erythroblasts, orthochromatic erythroblasts, reticulocytes, and mature red cells as confirmed by the characteristics of the sorted cells. Furthermore, sorting of murine bone marrow cells based on CD44 expression levels in conjunction with size change yielded nearly pure populations of different erythroblasts.

Disclosed herein is an ordered assembly of membrane proteins during erythropoiesis with increased levels of proteins important for mature red cell function and decreased levels of proteins not required or even harmful for mature red cell function. Moreover, use of α4 integrin in conjunction with band 3 enables the study of human erythropoiesis in vivo.

The surface expression of CD44 decreases by 30-fold in a stepwise manner in passing from the proerythroblast to the orthochromatic erythroblast in mice. In humans, this increase is seen in α4-integrin. The resulting ability to obtain, by cell sorting, highly purified populations of erythroblasts at all stages of maturation from primary bone marrow cells validated the choice of marker. By contrast, CD71, which has been in routine use as a surface marker for this purpose, has proved less effective. CD71 expression changes only fourfold and not in a progressive manner during terminal erythroid differentiation. This lack of significant decline in CD71 is physiologically relevant since uptake of transferring-bound iron is needed for heme synthesis at all stages of erythroid differentiation to sustain high levels of hemoglobin synthesis and as such little change in its expression is to be expected. \

The methods disclosed here in comprising contacting a sample comprising a population of erythroid cells with biomarker ligands specific for α4 integrin and band 3 to detect a level of expression of α4 integrin and band 3 on the erythroid cells, where in detection of a level of α4 integrin and band 3 identifies the stage of maturation of erythroid cells in the sample

As used herein, the term “biomarker ligand” refers to a molecule that can specifically bind to α4 integrin and band 3 expressed on erythroid cells or is differentially expressed on different subsets of erythroid cells. A biomarker ligand includes an antibody. As used herein, the term “antibody” refers to a molecule generated by an immune system that was made in response to a particular antigen that specifically binds to that antigen, and includes both naturally occurring antibodies and non-naturally occurring antibodies. For example, an antibody can be a polyclonal antibody, a monoclonal antibody, a dimer, a multimer, a multispecific antibody, a recombinant antibody, a humanized or primatized antibody, a chimeric antibody, bi-functional antibody, a cell-associated antibody like an Ig receptor, a linear antibody, a diabody, or a minibody, so long as the fragment exhibits the desired biological activity, and single chain derivatives of the same. An antibody can be a full-length immunoglobulin molecule comprising the VH and VL domains, as well as a light chain constant domain (CL) and heavy chain constant domains, CH1, CH2 and CH3, or an immunologically active fragment of a full-length immunoglobulin molecule, such as, e.g., a Fab fragment, a F(ab′)₂ fragment, a Fc fragment, a Fd fragment, a Fv fragment. An antibody can be derived from any vertebrate species (e.g., human, goat, horse, donkey, murine, rat, rabbit, or chicken), and can be of any type (e.g., IgG, IgE, IgM, IgD, and IgA), class (e.g., IgA, IgD, IgE, IgG, and IgM) or subclass (IgG1, IgG2, IgG3, IgG4, IgA1 and IgA2). For general disclosure on the structure of naturally occurring antibodies, non-naturally occurring antibodies, and antigenic compound-binding fragments thereof, see, e.g., Pluckthun in The Pharmacology of Monoclonal Antibodies, vol. 113, Rosenburg and Moore eds., Springer-Verlag, New York, pp. 269-315 (1994); Borrabeck, Antibody Engineering, 2d ed. (Oxford University Press 1995), each of which is hereby incorporated by reference in its entirety.

A biomarker ligand may be labeled or unlabeled. If labeled, the biomarker may be covalently or noncovalently attached with a fluorophore, a quantum dot, a phosphore, a chemiluminescent compound, a bioluminescent compound, a chromogenic compound, an isotope compound like a lanthanide, a radioisotope, a biotin or avidin molecule, or any other label useful for detecting a cell bound by the biomarker. Methods of attaching labels to antibodies are well known to those of ordinary skill in the art. Particularly preferred labels are those which are attached to the biomarker ligand by a linker which can be readily cleaved or separated or subject to hydrolysis by contact with a predetermined enzyme under physiological conditions. A biomarker ligand may be labeled before or after contact with the sample or before or after contact with the biomarker. A biomarker ligand may also be labeled by contacting with a labeled antibody which binds to the biomarker ligand. A biomarker ligand may also be conjugated with a magnetic particle, such as a paramagnetic nanoparticles (Miltenyi Biotec, Germany).

A sample comprising a population of erythroid cells as disclosed herein is contacted with a biomarker ligand. Contacting a sample with a biomarker ligand is done in such a way as to promote specific binding of the biomarker ligand to its cognate biomarker. Typically this is done under physiological conditions. For example, when a biomarker ligand is an antibody, contacting an antibody with a sample is done under conditions that result in the binding of the antibody to its corresponding biomarker, thereby resulting in an antibody/biomarker complex.

A sample comprising a population of erythroid cells as disclosed herein is screened to detect a level of cellular expression of the biomarkers. A labeled biomarker may be screened using a detection method based on fluorescence, bioluminescence, chemiluminescence, spectroscopic, photochemical, biochemical, immunochemical, chemical, magnetic, or other physical means known to one of ordinary skill in the art. An unlabeled biomarker may be screened using a detection method based on size, volume, density, opacity, or other physical means known to one of ordinary skill in the art.

In an embodiment, analyzing a sample comprising a population of erythroid cells to detect a level of cellular expression a biomarker is accomplished using a cell sorter. Cell sorters are well known to persons of ordinary skill in the art and generally are capable of separating a complex mixture of cells into fractions of a single cell type. Typically, the cells to be sorted are introduced as a thin jet of carrier liquid emanating from a small nozzle orifice. Shortly after leaving the nozzle, the hydrodynamically-focused stream of fluid passes through the waist of one or more tightly focused beams of light, usually laser light. A number of detectors are aimed at the point where the stream passes through the light beam: one in line with the light beam (Forward Scatter or FSC) and several perpendicular to it (Side Scatter or SSC) and one or more fluorescent detectors. Each suspended particle from 0.2 μm to 150 μm passing through the beam scatters the ray, and fluorescent chemicals found in the particle or attached to the particle may be excited into emitting light at a longer wavelength than the light source. This combination of scattered and fluorescent light is picked up by the detectors, and, by analyzing fluctuations in brightness at each detector (one for each fluorescent emission peak), it is then possible to derive various types of information about the physical and chemical structure of each individual particle. The data generated by flow cytometers can be plotted in a single dimension, to produce a histogram, or in two-dimensional dot plots or even in three dimensions. The regions on these plots can be sequentially separated, based on fluorescence intensity, by creating a series of subset extractions, termed “gates.” Some flow cytometers on the market have eliminated the need for fluorescence and use only light scatter for measurement. Other flow cytometers form images of each cell's fluorescence, scattered light, and transmitted light.

In an aspect of this embodiment, analyzing a sample comprising a population of erythroid cells to detect a level of a biomarker is accomplished by flow cytometer using a fluorescently-labeled biomarker ligand. Flow cytometric sorting is a specialized type of flow cytometry. It provides a method for sorting a heterogeneous mixture of biological cells into two or more containers, one cell at a time, based upon the specific light scattering and fluorescent characteristics of each cell. It is a useful scientific instrument, as it provides fast, objective and quantitative recording of fluorescent signals from individual cells as well as physical separation of cells of particular interest. A flow cytometric sorter can easily analyze cells at speeds greater than 200,000 events per second. Generally, the physics of the carrier fluid, however, and the statistics of distributing the cells among the droplets limits sort rates to about 50,000 cells per second. This combination of speed and reliable separation allows individual cells to be isolated or enriched for other uses.

In another embodiment, analyzing a sample comprising a population of T-cells to detect a level of cellular expression a biomarker is accomplished by magnetic-activated cell sorting (MACS) using a magnetically labeled biomarker ligand. MACS allows cells to be separated by incubating with magnetic nanoparticles coated with a biomarker ligand for a particular biomarker. Magnetic nanoparticles may comprise super-paramagnetic nanoparticles composed of iron oxide and a polysaccharide coat. The magnetic nanoparticles are preferably small enough to remain in colloidal suspension, which permits rapid, efficient binding to cell surface biomarker. In aspects of this embodiment, the magnetic nanoparticles are between about 1 nm in diameter to about 100 nm in diameter, such as, e.g., about 25 nm in diameter, 50 nm in diameter, 75 nm in diameter, or 100 nm in diameter. In other aspects of this embodiment, the magnetic nanoparticles have a volume of, e.g., about one-millionth that of a typical mammalian cell, about five-millionth that of a typical mammalian cell, or about ten-millionth that of a typical mammalian cell. The magnetic nanoparticles preferably do not interfere with flow cytometry, are biodegradable, and have negligible effects on cellular functions. The antibody coupling to the magnetic nanoparticles may be direct or indirect, via a second antibody to a ligand such as a fluorophore, a quantum dot, a phosphore, a chemiluminescent compound, a bioluminescent compound, a chromogenic compound, an isotope compound like a lanthanide, a radioisotope, an enzyme, a biotin or avidin molecule, or any other label useful for detecting a cell bound by the biomarker.

In another embodiment, analyzing a sample comprising a population of erythroid cells to detect a level of a biomarker is accomplished by solid-phase attachment. In an aspect, screening a sample comprising a population of erythroid cells to detect a level of a biomarker is accomplished by panning or solid-phase affinity chromatography using a biomarker ligand as disclosed herein. In an aspect, analyzing a sample comprising a population of erythroid cells to detect a biomarker is accomplished by solid-phase magnetic beads using a magnetically labeled biomarker ligand as disclosed herein. See, e.g., US Patent Application Publication 2005/0186207, which is incorporated by reference in its entirety.

In another embodiment, analyzing a sample comprising a population of erythroid cells to detect a level of a biomarker is accomplished by complement cell lysis. Complement cell lysis is used to eliminate undesired cell population recognized by antibodies, and is based in the function of certain type of antibodies to fix and activate a cascade of enzymatic molecules called “Complement system” on cell surface. Final reaction result in opening a physical hole in the membrane and produce cell lysis by osmosis. Typically, cells are incubated during 30 minutes at 4° C. with an antibody and a source of complement enzymes is added and incubated at 37° C. Finally, cells are washed with isotonic buffers and ready to be used.

A population of erythroid cells is identified based on a characteristic expression pattern of α4 integrin and band 3. Generally, such cells are identified according to the expression levels biomarkers based upon readily discernible differences in staining intensity as is known to one of ordinary skill in the art. Typically, the expression of a biomarker is classified as high (biomarker^{hi}), +(biomarker⁺), low (biomarker^{low}) and −(biomarker⁻).

Cells staining intensely or brightly when screened using a biomarker ligand is referred to as biomarker⁺, and is indicative of a cell exhibiting a high level of biomarker expression. Cells staining slightly, dully, or not at all when screened using a biomarker ligand is referred to as biomarker^{low/-}, and is indicative of a cell exhibiting a high level of biomarker expression.

The cut off for designating a cell as a biomarker^{hi} cell can be set in terms of the fluorescent intensity distribution observed for all cells with those cells in the top 2%, 3%, 5%, 7% or 10% of fluorescence intensity being designated as biomarker^{hi} cells. The cut off for designating a cell as a biomarker⁺ cell can be set in terms of the fluorescent intensity distribution observed for all cells with those cells in the top 10%, 20%, 30%, 40% or 50% of fluorescence intensity being designated as biomarker⁺ cells.

The cut off for designating a cell as a biomarker^{low} cell can be set in terms of the fluorescent intensity distribution observed for all cells with those cells falling below 50%, 40%, 30%, 20%, or 10% fluorescence intensity being designated as biomarker^{low/-} cells. The cut off for designating a cell as a biomarker⁻ cell can be set in terms of the fluorescent intensity distribution observed for all cells with those cells falling below 10%, 7%, 5%, 3%, or 2% fluorescence intensity being designated as biomarker⁻ cells.

Cells may also be distinguished by obtaining the frequency distribution of biomarker staining for all cells and generating a population curve fit to a higher staining population and a lower staining population. Individual cells are then assigned to the population to which they are likely to belong based upon a statistical analysis of the respective population distributions. In one embodiment, biomarker^{low/-} cells exhibit one-fold or less, two-fold or less, or three-fold less fluorescence intensely than biomarker⁺ cells.

The screening and contacting steps are as described herein.

A population of erythroid cells is isolated or enriched based on a characteristic expression pattern of α4 integrin and band 3. As for the analyzing step disclosed herein, such cells are isolated or enriched according to the expression levels of α4 integrin and band 3 based upon readily discernible differences in staining intensity as is known to one of ordinary skill in the art. Typically, the expression of a biomarker expression pattern is classified as high (biomarker^{hi}), +(biomarker⁺), low (biomarker^{low}) and −(biomarker⁻).

A population of the desired erythroid cells may be comprised substantially of cells comprising the desired biomarker expression pattern. As used herein, the term “substantially”, when used in reference to a population of cells comprising the desired biomarker expression pattern refers to a population of cells for which at least 80% of the total number of cells from the population comprises the desired biomarker expression pattern. In aspects of this embodiment, a population of erythroid cells comprising the desired α4 integrin and band 3 expression pattern makes up, e.g., at least 83%, at least 85% at least 88%, or at least 90%, at least 93%, at least 95% at least 98%, or at least 99% of the total number of cells from the population. In other aspects of this embodiment, a population of erythroid cells comprising the desired α4 integrin and band 3 expression pattern is makes up, e.g., at least two-fold, at least four-fold, at least eight-fold, at least ten-fold, at least 20-fold, at least 50-fold, or at least 100-fold as compared to the source population of the desired erythroid cells from the sample.

A population of erythroid cells may be isolated or enriched using positive selection or negative selection of the cells of interest. Additionally, a population of erythroid cells may be isolated or enriched using both positive selection and negative selection of the cells of interest. As used herein, the term “positive selection” refers to the selection of specified cells from a mixture or starting population of cells based upon the high or positive expression of a biomarker on the specified cells. As used herein, the term “negative selection” refers to the selection of specified cells from a mixture or starting population of cells based upon the low or negative expression of a biomarker on the specified cells. The biomarkers used for positive or negative selection of cells may be detected by, e.g., flow cytometric sorter, MACS, solid-phase attachment, panning, and chromatography. Immunoselection of α4 integrin and band 3 on cells may be performed in one or more steps, wherein each step positively or negatively selects for one or more biomarkers. When immunoselection of two or more biomarkers is performed in one step using flow cytometric sorter, the two or more different biomarkers may be labeled with different fluorophores.

In aspects of this embodiment, a population of erythroid cells comprising the desired α4 integrin and band 3 expression pattern is enriched by, e.g., at least 20%, at least 30%, at least 40% at least 50%, or at least 60%, at least 70%, at least 80%, or at least 90% as compared to the total number of cells from the source population of cells. In other aspects of this embodiment, a population of erythroid cells comprising the desired α4 integrin and band 3 expression pattern is enriched by, e.g., at least two-fold, at least four-fold, at least eight-fold, at least ten-fold, at least 20-fold, at least 50-fold, or at least 100-fold as compared to the total number of cells from the source population of cells.

In other aspects of this embodiment, a population of erythroid ells comprising the desired α4 integrin and band 3 expression pattern is enriched to, e.g., at least 80%, at least 85%, at least 90%, at least 95%, or at least 98% of the total number of cells in the sample. In yet other aspects of this embodiment, a population of erythroid cells comprising the desired α4 integrin and band 3 expression pattern is enriched to, e.g., about 80% to about 85%, about 80% to about 90%, about 80% to about 95%, about 80% to about 98%, about 80% to about 100%, about 85% to about 90%, about 85% to about 95%, about 85% to about 98%, about 85% to about 100%, about 90% to about 95%, about 90% to about 98%, or about 90% to about 100%, of the total number of cells in the sample.

In another aspect, a population of erythroid cells comprising the desired α4 integrin and band 3 expression pattern is isolated by a negative selection scheme that depletes undesired cells from the cells in the sample.

In an embodiment, isolating a subpopulation of T-cells comprising a desired α4 integrin and band 3 expression pattern is accomplished using a cell sorter as disclosed herein. In an aspect of this embodiment, isolating a subpopulation of erythroid cells comprising a desired α4 integrin and band 3 expression pattern is accomplished using flow cytometric sorter as disclosed herein. In another embodiment, isolating a subpopulation of erythroid cells comprising a desired α4 integrin and band 3 expression pattern is accomplished using MACS as disclosed herein. A biomarker expression pattern may be used for the positive selection or the negative selection of cells of interest as disclosed herein. The biomarkers expression pattern used for positive or negative selection of cells may be detected by, e.g., flow cytometric sorter, MACS, panning, and chromatography.

The desired α4 integrin and band 3 expression pattern is dependent on the stage of cell to be analyzed or isolated.

There is no limitation on the biological samples that can be used in the methods disclosed herein. In one non-limiting example, the sample is from blood, bone marrow, cord blood, placenta or spleen. Additionally, the sample can be from an in vitro or ex vivo culture of cells. In other embodiments, the sample can be from blood donors on whom apheresis has been performed in order to collect adult stem cells from the peripheral blood and/or from processing methods in which stem cells are being induced to mature into erythrocytes.

### EXAMPLES

### Example 1

### Ordered Assembly of Membrane Proteins during Differentiation of Murine Erythroblasts

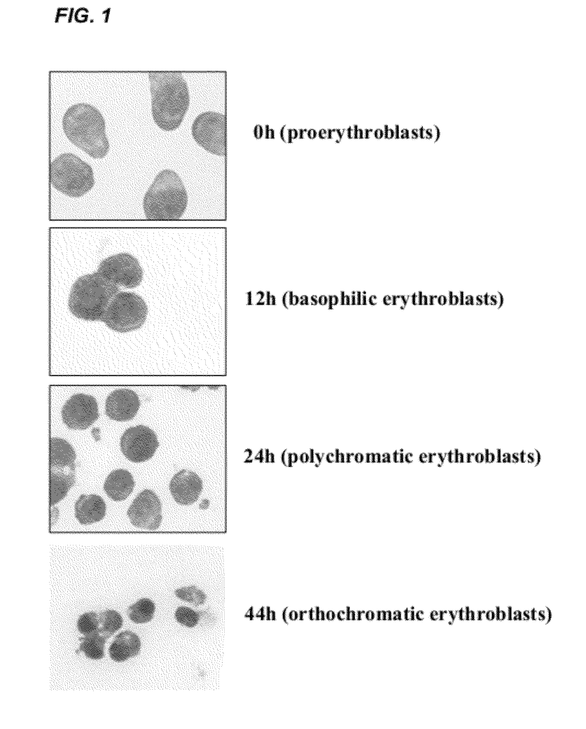

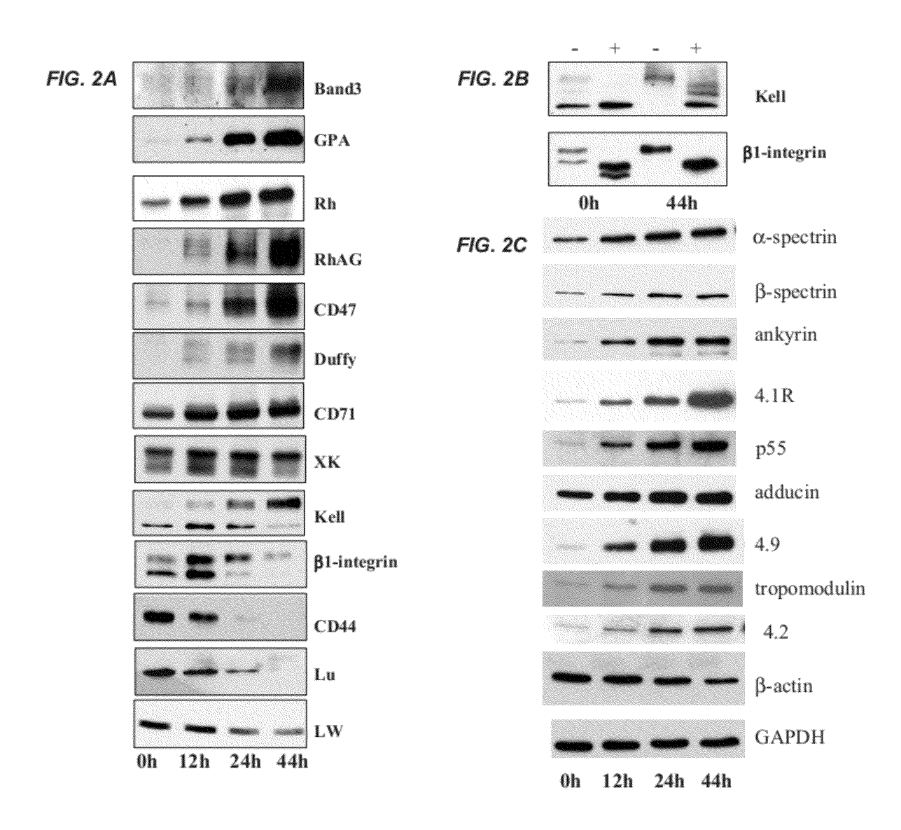

It is expected that the differentiation of erythroblasts would be accompanied by the changes in protein composition and properties of the plasma membrane, however a comprehensive study has not been reported. Using FVA system (Koury et al. J. Cell Physiol. 121:526-63, 1984) near homogenous populations of erythroblasts were obtained at proerythroblast, basophilic erythroblast, polychromatic erythroblast, orthochromatic erythroblast stages (depicted in FIG. 1) and the expression of 13 transmembrane proteins examined by Western blotting. During 44 hours of culture in this system, proerythroblasts (0 hr) progressively differentiated into basophilic erythroblasts (12 hr), polychromatic erythroblasts (24 hr), and orthochromatic erythroblasts and reticulocytes (44 hr). FIG. 2A depicts the relative concentrations of these proteins as assessed by Western blotting. It revealed following changes: 1) the major red cell proteins band 3, GPA, GPC, Rh, RhAG, Duffy, and CD47 were expressed at low level in proerythroblasts but were significantly increased in late stage erythroblasts; 2) by contrary, adhesion molecules β1 integrin, CD44, Lu, and ICAM-4 were expressed at the highest level in proerythroblasts and were decreased in late stage erythroblasts; 3) the transferrin receptor (CD71) and XK were slightly increased in the progression from proerythroblasts to basophilic erythroblasts.

Two distinct immunoreactive protein bands were observed for Kell and β1 integrin (FIG. 2A) and N-glycosidase treatment was performed to determine if the two bands reflect differences in glycosylation or expression of different isoforms (FIG. 2B). While this did not change the migration of the lower 94 kDa band of Kell in proerythroblasts (0 hr), the upper band (130 kDa) expressed in orthochromatic erythroblasts (44 hr) decreased in apparent size, implying that the unglycosylated 94 kDa component is expressed in proerythroblasts while the glycosylated 130 kDa component was expressed in later stages of erythroid differentiation. Two β1 integrin bands were seen in proerythrocytes (0 hr), both of which shifted after N-glycosidase treatment, implying that both are expressed at this stage and are glycosylated. Only the glycosylated higher molecular weight isoform was expressed at low levels in orthochromatic erythroblasts (44 hr).

To fully understand the molecular changes of membrane proteins during erythropoiesis, cytoskeletal protein compositions were also compared in various stages of erythropoiesis. The expression levels of 10 skeletal proteins during terminal erythroid differentiation determined by Western blotting are shown in FIG. 2C. In contrast to the three distinct patterns of expression of transmembrane proteins, all skeletal proteins, with the exception of actin, adhered to a single pattern of expression. The expression of α-spectrin, β-spectrin, ankyrin, 4.1R, 4.2, p55, tropomodulin, dematin, and adducin, increased during terminal differentiation, whereas that of actin decreased in late-stage erythroblasts compared to proerythroblasts.

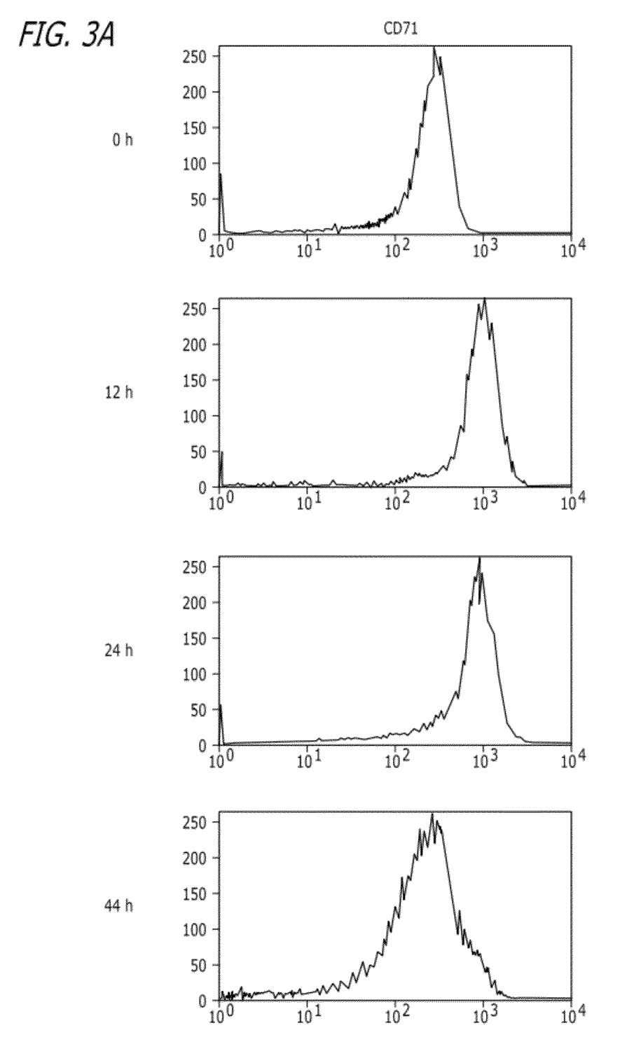

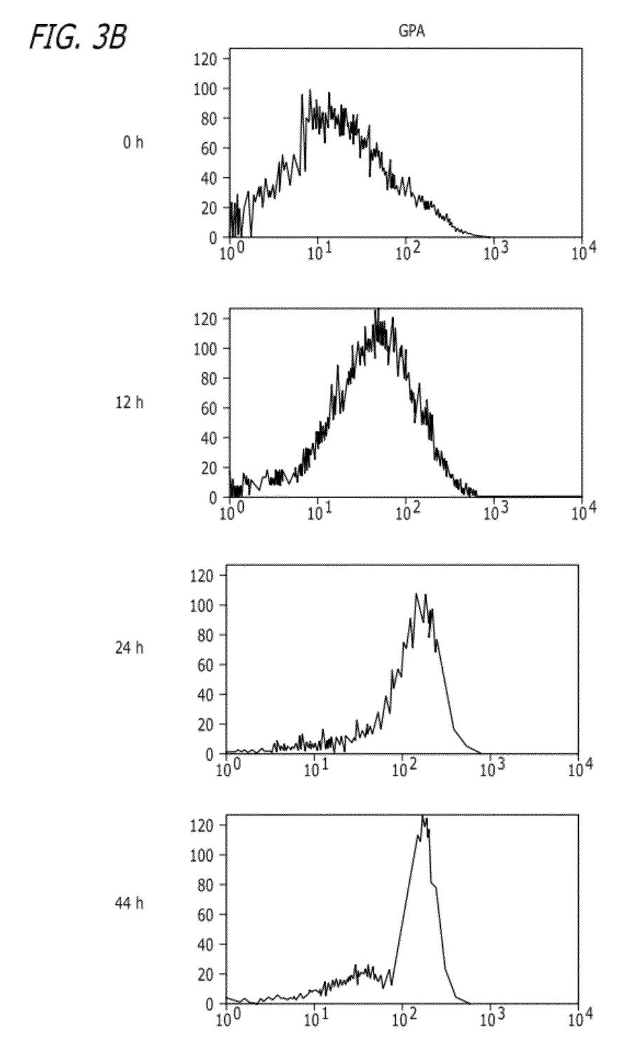

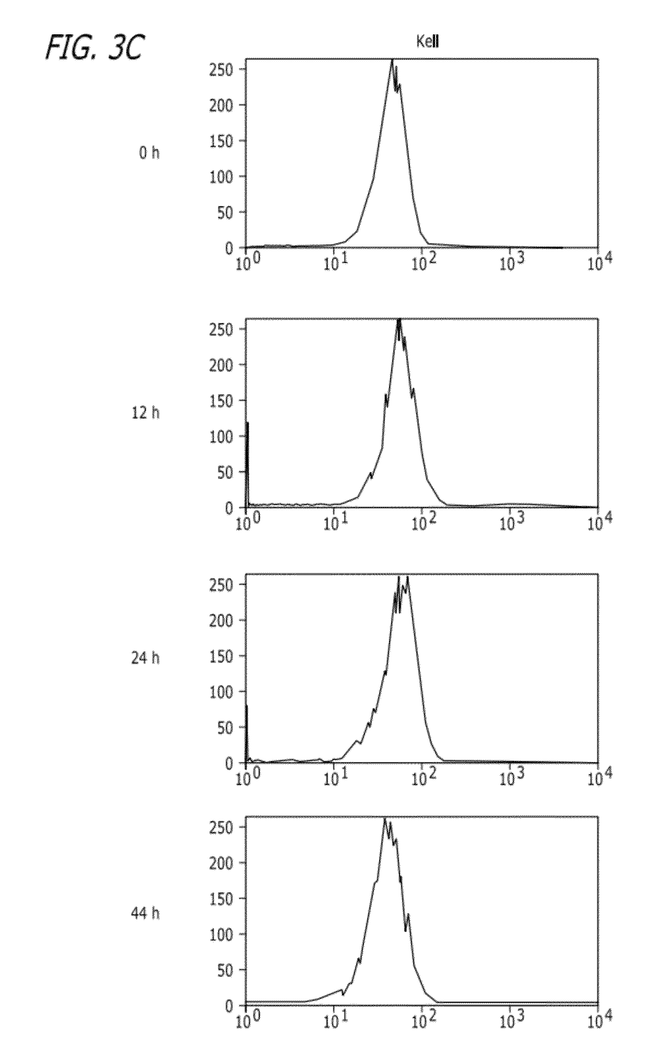

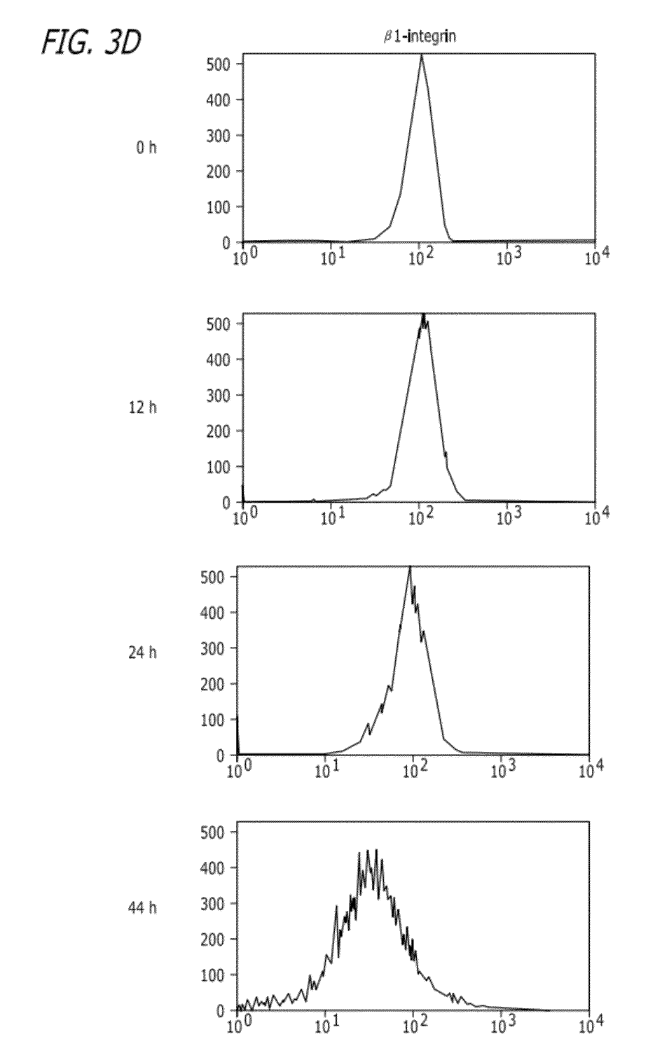

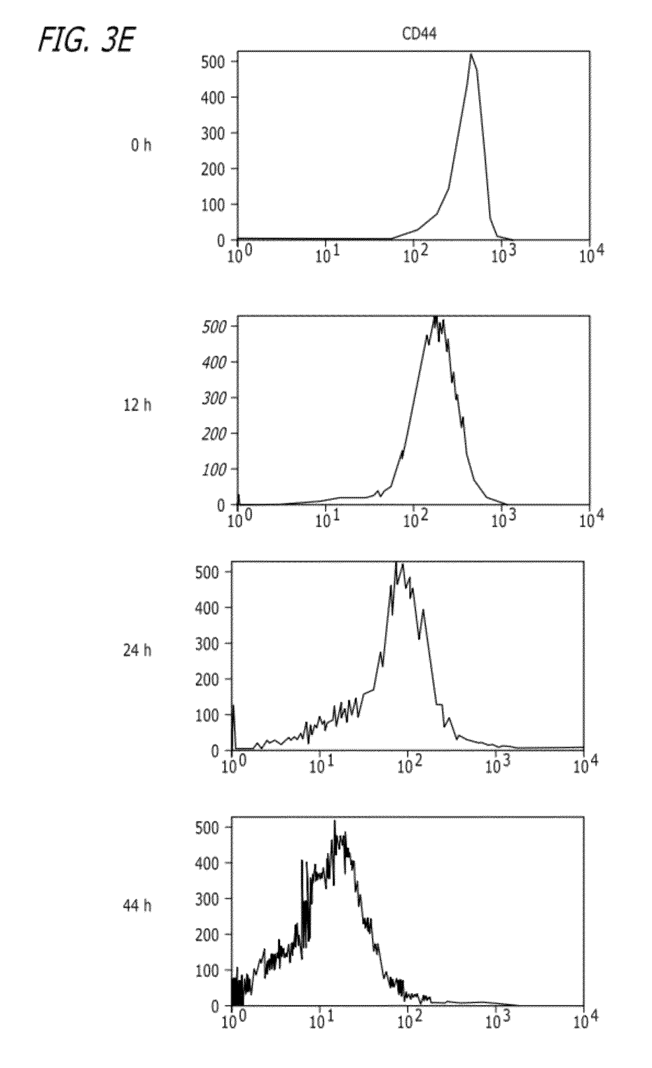

Since Western blot analysis provided total protein content, the surface expression of some transmembrane proteins including CD71, GPA, Kell, β1 integrin, and CD44 was further examined by flow cytometry. As shown in FIG. 3 and Table 1, the surface expression of CD71 increased three- to four-fold from proerythroblasts to basophilic and polychromatic erythroblasts. However, late-stage orthochromatic erythroblasts expressed similar levels of surface CD71 as that of proerythroblasts. Glycophorin A exhibited a progressive increase, with four times greater abundance in orthochromatic erythroblasts as compared to proerythroblasts. No significant change was observed for surface expression of Kell. As for β1 integrin, in contrast to the results of Western blot analysis, which showed an increase from proerythroblasts to basophilic erythroblasts, followed by a progressive decrease in late stages, surface expression of β1-integrin was significantly decreased only in orthochromatic erythroblasts. The most dramatic change occurred in surface expression of CD44, which decreased more than 30-fold from proerythroblast to orthochromatic erythroblasts. The mean fluorescence intensity of unstained cells as well as cells stained with secondary antibody was less than 100.

TABLE 1
Expression of Surface Markers During Erythropoiesis

| Proteins | 0 hr | 12 hr | 24 hr | 40 hr |
|---|---|---|---|---|
| CD71 | 6700 ± 100 | 26000 ± 730 | 22000 ± 450 | 6500 ± 180 |
| GPA | 1000 ± 80 | 1800 ± 50 | 3500 ± 130 | 3700 ± 100 |
| Kell | 1300 ± 15 | 1600 ± 30 | 1600 ± 40 | 1170 ± 30 |
| β1 | 2500 ± 130 | 2880 ± 180 | 2360 ± 140 | 760 ± 25 |
| integrin | | | | |
| CD44 | 8900 ± 400 | 4870 ± 160 | 2070 ± 40 | 270 ± 10 |

The expression of GPA, band 3, RhAG, CD71, and CD44 was then examined in primary mouse bone marrow erythroblasts by immunofluorescence microscopy. Erythroblasts were defined by positive staining for GPA and with 4′6-diamidino-2-phenylindole (DAPI). The early- and late-stage erythroblasts were distinguished based by cell size, large cells representing an early-stage, and small ones late-stage erythroblasts. As shown in FIG. 2a, an increase in cell surface expression of GPA, band 3, and RhAG from early- to late-stage erythroblasts was readily apparent. In marked contrast, there was a dramatic decrease in surface expression of CD44. Little change in the surface expression of CD71 was noted between early- and late-stage erythroblasts. These findings are consistent with the results on cultured erythroblasts.

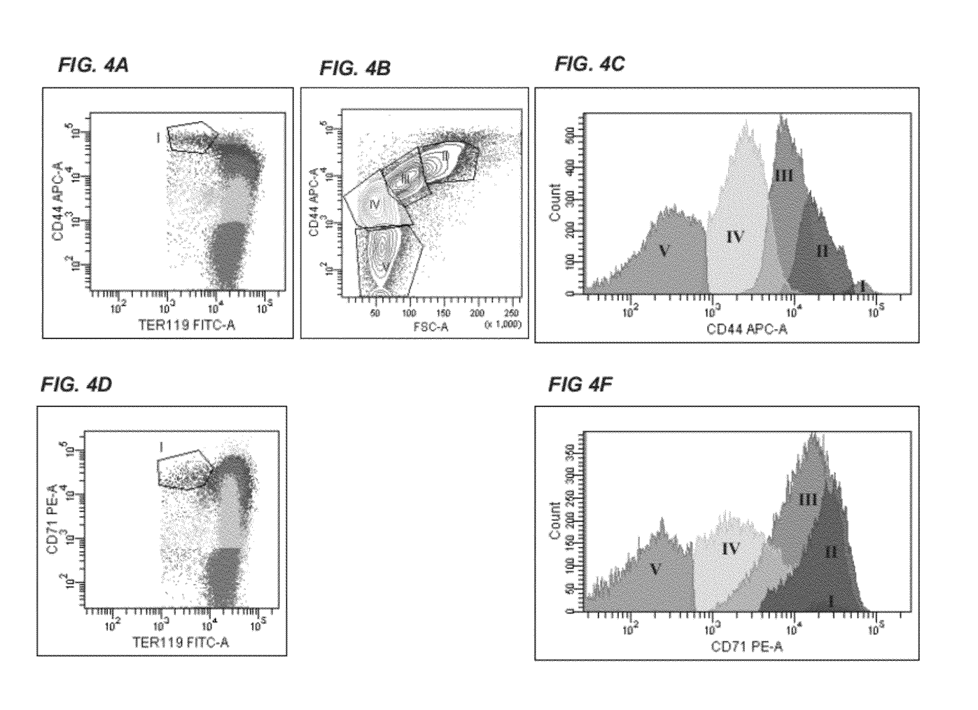

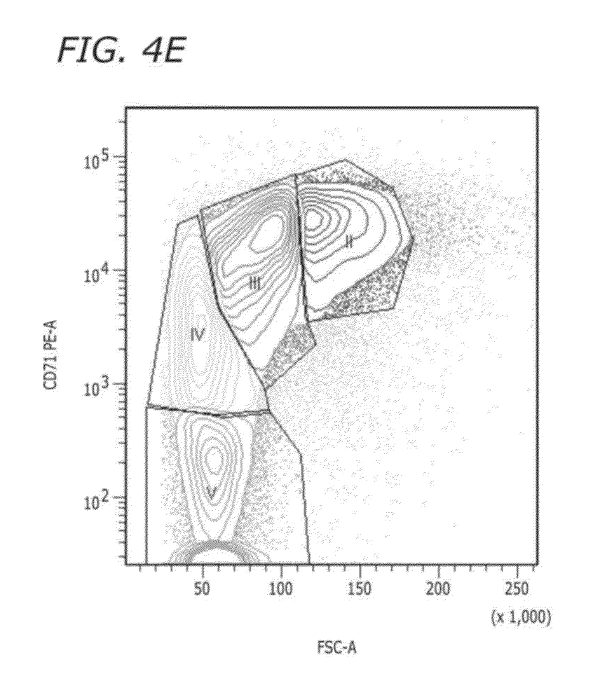

CD71 in conjunction with TER119 has been used as a surface marker to distinguish different stages of erythroblasts in vivo based on the assumption that CD71 decreases significantly during erythropoiesis. However, the present inventors have demonstrated that both total and surface expression of CD71 does not have significant changes during erythropoiesis. Instead, the total as well as the surface expression of CD44 demonstrated a progressive reduction from stage to stage and decreased more than 30-fold from proerythroblast to orthochromatic erythroblast. These findings suggest that CD44 is a more reliable surface marker for distinguishing between different stages of murine erythroid differentiation than CD71. To confirm this observation, bone marrow cells were stained with both CD44 and an erythroid-specific glycophorin A antibody, TER119. FIG. 4A depicts a plot of CD44 versus TER119. Based on the TER119 staining intensity, two distinct populations, TER^{low} and TER^{hi}, were seen. The terms “low” and “hi” when used relative to cell surface expression of proteins refers to relative levels of expression. To further distinguish erythroblast subpopulations, forward scatter (FSC) intensity was used since FSC is a function of cell size and erythroblasts decrease in size with maturation. FIG. 4B depicts expression levels of CD44 as a function of FSC for all TER119 positive cells. Five distinct clusters can be seen. The histographic presentation of CD44 expression levels in the five gated cell populations (FIG. 4C) shows progressive decrease of CD44 surface expression with decreased cell size.

In parallel, bone marrow cells were also stained with TER119 and CD71 and the data analyzed in a similar manner (FIG. 4D-4F). Based on TER119 staining intensity, two distinct populations TER^{low} and TER^{hi} were once again seen (FIG. 4D). However, when CD71 expression levels were analyzed as a function of FSC for all TER⁻ positive cells (FIG. 4E), there was a marked overlap in the histogram profiles of CD71 between the gated clusters I to III, implying similar levels of CD71 (FIG. 4F).

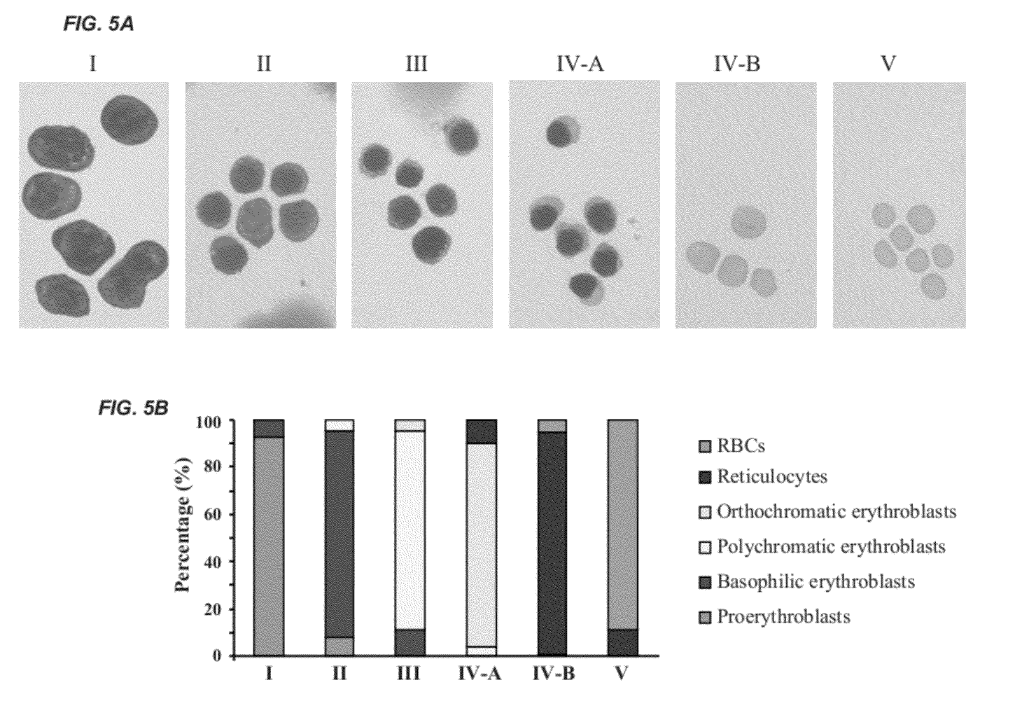

To identify different erythroblast populations, primary bone marrow erythroid cells were sorted based on either CD44 or CD71 expression levels and cell size. FIG. 5A depicts representative images from each of the five CD44 stained populations. Cells from region I have morphological characteristics of proerythroblasts, namely large size, very high nucleus/cytoplasm ration and intensely basophilic cytoplasm. Cells from region II are smaller in size, with increased nuclear condensation and the morphological characteristics of basophilic erythroblasts. Cells from region III are polychromatic erythroblasts, exhibiting the further decrease in cell size and additional nuclear condensation. Initial sorting of the region IV population showed mixed populations of orthochromatic erythroblasts and immature reticulocytes. Region IV cells were thus gated into two distinct populations based on the expression levels of CD44, termed IV-A (higher CD44 expression, top half of region IV) and IV-B (lower CD44 expression, bottom half of region IV). As shown in FIG. 5A, cells from region IV-A have cellular characteristics of orthochromatic erythroblasts while cells from region IV-B are non-nucleated reticulocytes. Finally, cells from region V were predominantly mature red cells. To quantify the purity of the various sorted populations, a differential count of erythroblasts at different stages of development was performed by examining 1000 cells. As shown in FIG. 5B, the various sorted populations contained cells at a defined stage of development ranging from proerythroblasts to reticulocytes with purities ranging from 85 to 90%.

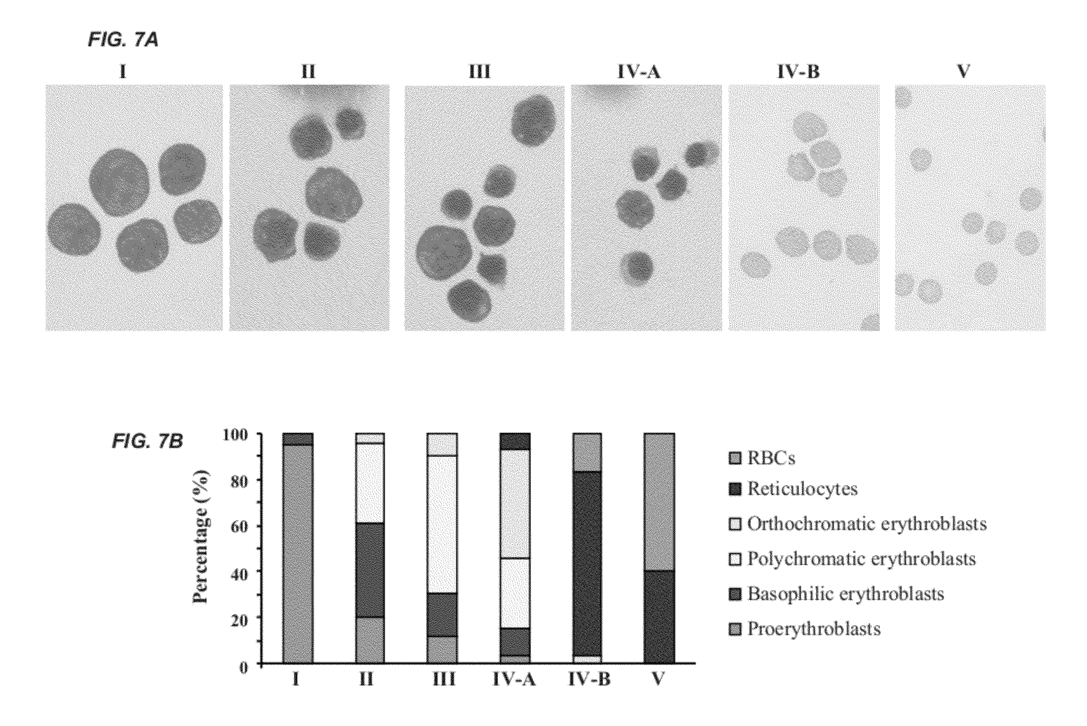

Representative images of erythroblast morphology on stained cytospins of each of the five CD71 stained populations, are shown in FIG. 7A. While, as with CD44, more than 90% of cells from region I were proerythroblasts, there was large degree of heterogeneity in all other regions (FIG. 7B). The purity of erythroblasts at all later stages of development ranged between 40 to 60% in the different fractions.

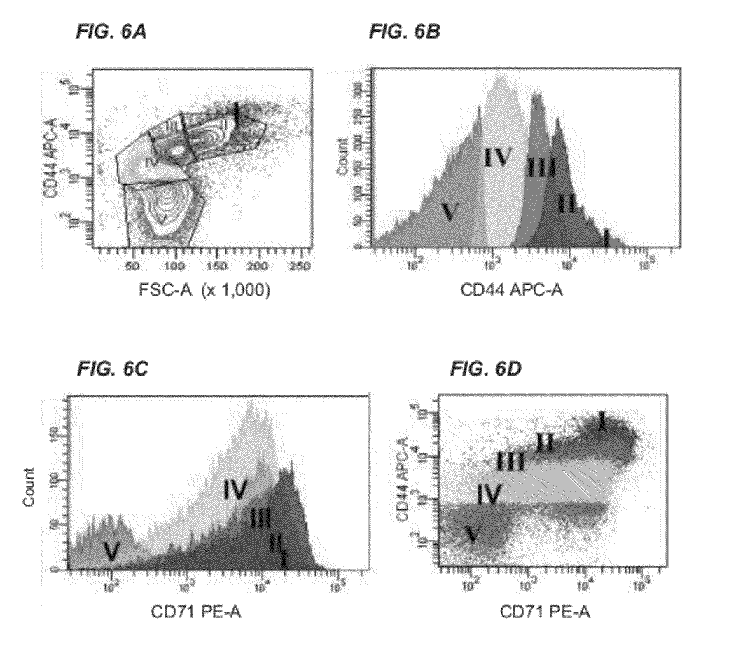

To further validate that CD44 is a more effective surface marker for distinguishing murine erythroblasts at different stages of erythroid differentiation than CD71, expression levels of CD44 and CD71 were compared on the same cells were measured by dual staining of primary bone marrow cells with both antibodies along with TER119. As shown in FIGS. 6A and 6B, gating on five distinct forward scatter gates of the dual stained cells, identified erythroblasts with five distinct levels of CD44 expression, consistent with staining with CD44 staining alone. In marked contrast, there was significant overlap in CD71 expression levels in the same five gated populations (FIG. 6C). Moreover, as shown in FIG. 6D, there is a wide range of CD71 expression levels at several maturation stages compared to CD44, confirming CD71 does not change progressively and distinctly during terminal erythroid differentiation.

Methods

Antibodies. For western blot, most antibodies were generated and characterized as previously described (Salomao et al. Proc. Natl. Acad. Sci. USA 105:8026-31, 2008). Anti-TER119, anti-β1 integrin, and anti-CD44 were obtained from BD Pharmingen. Anti-CD71 was from Invitrogen. For flow cytometry and sorting, the antibodies used were as follows: FITC-conjugated anti-TER119, APC-conjugated anti-CD44, PE-conjugated anti-CD71, APC-Cy™ 7-conjugated CD11b, all of which were from BD Pharmingen, FITC-conjugated anti-β1 integrin was from BioLegend and monoclonal anti-Kell was generated internally.

Erythroid Cultures. Proerythrocytes were isolated and cultured using methods previously established (Koury et al. J. Cell Physiol. 121:526-32, 1984; Bondurant et al. Blood 61:751-8, 1983). Briefly, two weeks after infection with 10⁴ spleen focus-forming units of the anemia-inducing strain of Friend leukemia virus (FVA), female CDF-1 mice (Charles River) were sacrificed and splenic proerythroblasts were purified by velocity sedimentation at unit gravity. The proerythroblasts were cultured at 37° C. in a humidified atmosphere of 5% CO₂ in air at a cell concentration of 1×10⁶ cells/mL in Iscoves-modified Dulbecco medium (IMDM; Gibco) with 30% heat-inactivated, fetal bovine serum (Invitrogen), 1% deionized bovine serum albumin (Millipore), 100 units/mL penicillin G (ATCC), 100 g/mL streptomycin (ATCC), 0.1 mM α-thioglycerol (Sigma), and 0.2 units/mL human recombinant erythropoietin (EPO; R&D). Erythroblasts of different stages were collected at 12 hr, 24 hr and 44 hr.

Western blot analysis. Whole-cell lysates of erythroid cells were prepared with RIPA buffer (150 mM NaCl, 1.0% Nonidet P-40, 0.5% sodium deoxycholate, 0.1% SDS, 1 mM EDTA, and 50 mM Tris HCl, pH 8.0) in the presence of protease inhibitor cocktails (Sigma). Protein concentration was measured using the DC protein assay kit (BioRad). For N-glycosidase treatment, 40 μg total protein diluted in 50 μL lysis buffer were digested with or without 5 unit N-glycosidase F (NglyF, Sigma) for 16 hr at 37° C. Thirty micrograms of protein were run on 10% SDS/PAGE gels and transferred to nitrocellulose membrane (BioRad) for 2 hr at 60V. The membrane was blocked for 1 hr in PBS containing 5% nonfat dry milk, and 0.1% Tween-20, and then probed for 2 hr with primary antibody diluted in 5% nonfat milk and 0.1% Tween-20. After several washes, blots were incubated with secondary antibody coupled to HRP (Jackson Laba) diluted in 5% nonfat milk and 0.1% Tween-20, washed, and developed on Kodak BioMax MR film (Sigma) using the SuperSignal West Pico Chemiluminescent Substrate (Thermo Scientific).

Immunofluorescence microscopy. Bone marrow cells were washed 3 times by centrifugation in PBS/0.5% BSA, and allowed to adhere for 1 hr to slides coated with Cell-Tak (BD Pharmingen). The cells were washed 3 times in PBS, fixed with 4% paraformaldehyde, 0.2% Triton X-100 in PBS at room temperature for 10 minutes, further permeabilized by 1% Triton X-100 in PBS for 3 min, and then rinsed with PBS. After incubation in 10% donkey serum (Jackson ImmunoResearch) and 10 mg/mL bovine serum albumin (BSA; Sigma) in PBS for 1 hr to block nonspecific protein binding, fixed cells were treated with primary antibodies diluted in 10 mg/mL PBS/BSA for 1 hr at room temperature. Cells were washed at 5 min intervals 6 times with gentle shaking, incubated for 1 hr with secondary antibodies at 1:700 in 10 mg/mL PBS/BSA, washed 3 times in PBS; then mounted using Vectashield with DAPI (Vector Laboratories). Fluorescence was imaged using a Zeiss Axiovert 135 microscope with a 63×/1.25 oil immersion objective and equipped with a CCD camera.

Flow cytometric analysis of cultured cells and primary bone marrow cells. Cultured erythroblasts (1×10⁶) were stained with PE-conjugated CD71, APC-conjugated CD44, FITC-conjugated TER119, FITC-conjugated β1 integrin, or unconjugated anti-Kell in PBS/0.5% BSA for 20 min at 4° C. Then the cells were washed twice in PBS/0.5% BSA. For Kell, the cells were then incubated for 20 min with goat anti-mouse IgG-FITC (Invitrogen) and the cells were washed twice in PBS/0.5% BSA and the surface antigen expression of these proteins was analyzed. Bone marrow cells were harvested from the tibia and femur of mice (3 months old). For phenotype analysis by flow cytometry, 2×10⁶ cells were re-suspended in 80 μL PBS/0.5% BSA. Cells were blocked with rat anti-mouse CD16/CD32 (5 μg/10⁶ cells) for 15 min. Then, samples were stained with FITC rat anti-mouse TER119 (1 μg/10⁶ cells), APC rat anti-mouse CD44 (1 μg/10⁶ cells), and APC-Cy™ 7 rat anti-mouse CD11b (0.3 μg/10⁶ cells) on ice for 20-30 min in the dark. Cells were washed twice with 700 μL PBS/0.5% BSA. Finally, cells were re-suspended in 100 μL PBS/0.5% BSA and stained with the viability marker 7-AAD (0.25 μg/10⁶ cells) on ice for 10 min in the dark. Cells were then suspended in 0.4 mL of PBS/0.5% BSA and analyzed within one hour following staining using BD FACSDiva™ software on FACSCanto™ flowcytometer. Unstained cells or cell stained with second antibody only (in the case of Kell staining) were used as negative controls. Mean fluorescent intensity (FLI) was used as a measure of antibody binding.

Fluorescence activated cell sorting. To isolate erythroblasts at different stages of maturation by cell sorting, 200×10⁶ cells were re-suspended in 8 mL PBS/0.5% BSA in a 50 mL tube. Cells were blocked with rat anti-mouse CD16/CD32 (5 μg/10⁶ cells) for 15 min. Then, samples were stained with FITC rat anti-mouse TER119 (1 μg/10⁶ cells), APC-Cy™ 7 rat anti-mouse CD11b (0.3 μg/10⁶ cells) and APC rat anti-mouse CD44 (1 μg/10⁶ cells) or with PE rat anti-mouse CD71 (1 μg/10⁶ cells) instead of CD44 and incubated on ice for 20-30 min in the dark. Cells were washed twice with 40 mL PBS/0.5% BSA and re-suspended in 10 mL PBS/0.5% BSA and stained with the viability marker 7-AAD (0.25 μg/10⁶ cells) on ice for 10 min in the dark. Sorting was performed on a MOFLO™ high speed cell sorter (Beckman-Coulter).

Cytospins. For determining cell morphology, 100 μL of cell suspension containing 10⁵ sorted cells were used to prepare cytospin preparations on coated slides using the Thermo Scientific Shandon 4 Cytospin. The slides were stained with May-Grunwald (Sigma) solution for 5 min, rinsed in Tris buffer pH7.2, for 90 sec and subsequently stained with Giemsa solution (Sigma).

### Example 2

### Quantitative Analysis of Erythropoiesis in Mouse Bone Marrow

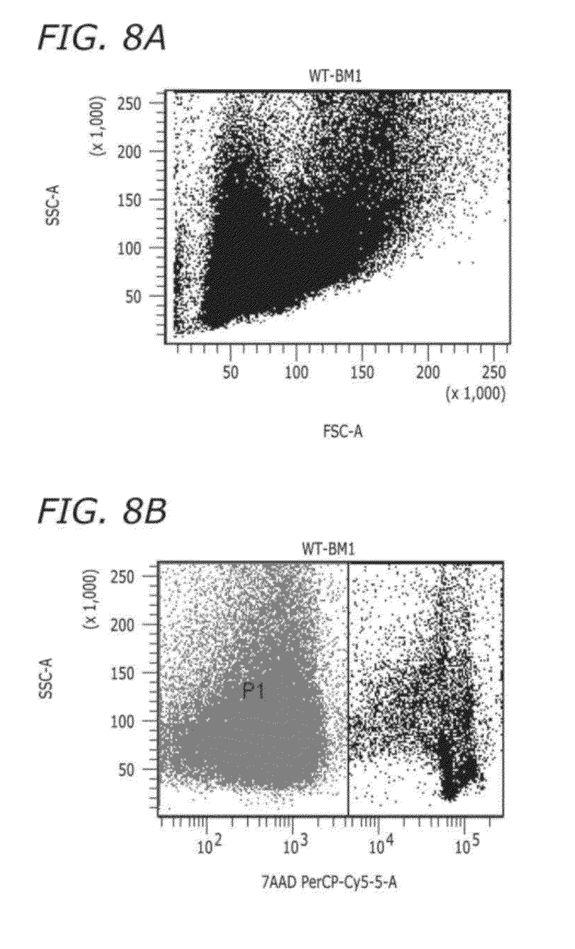

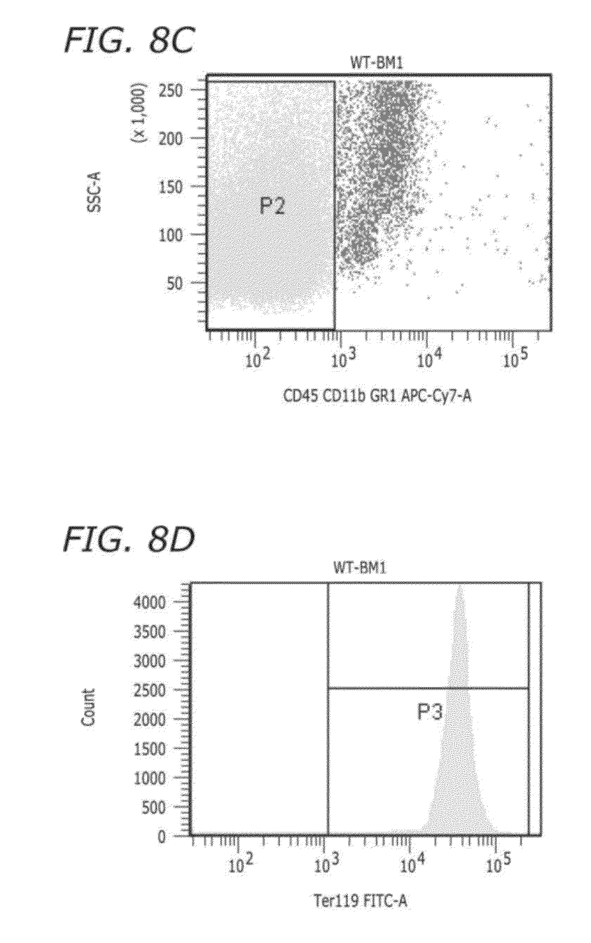

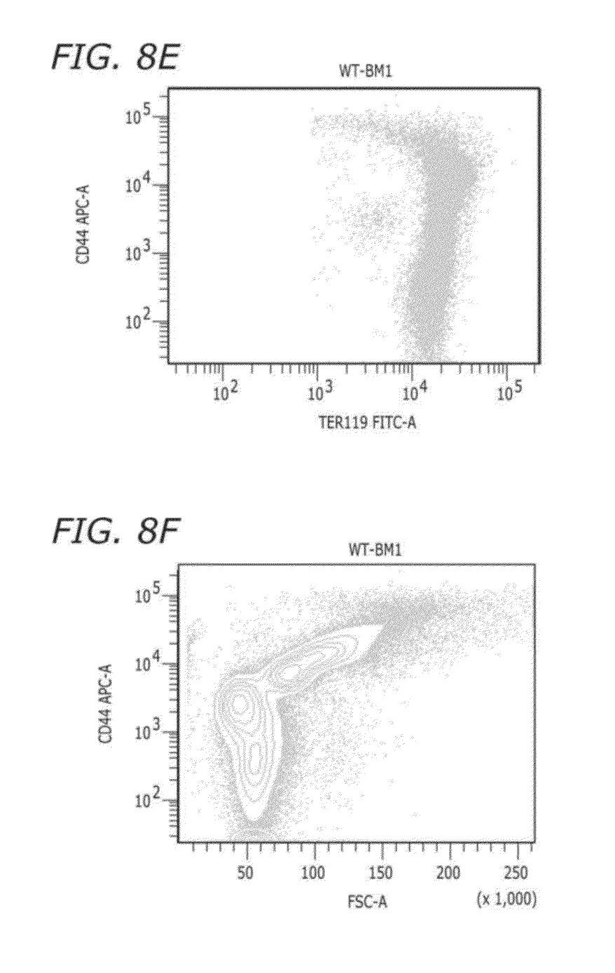

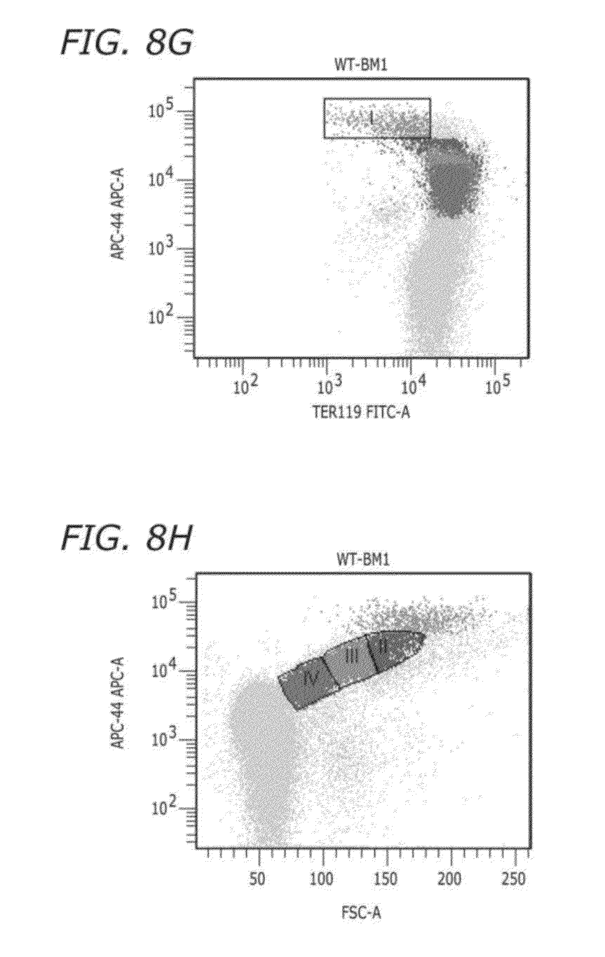

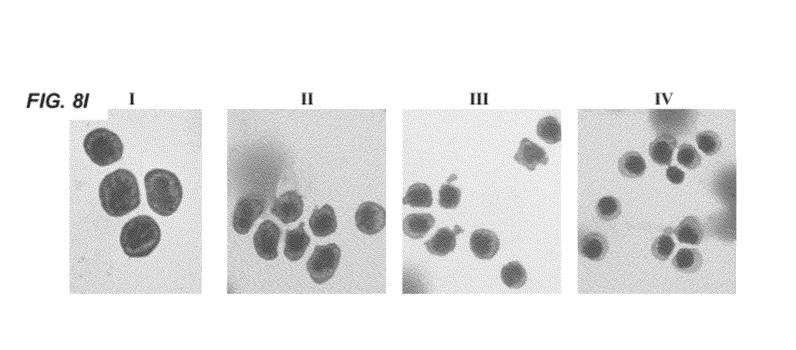

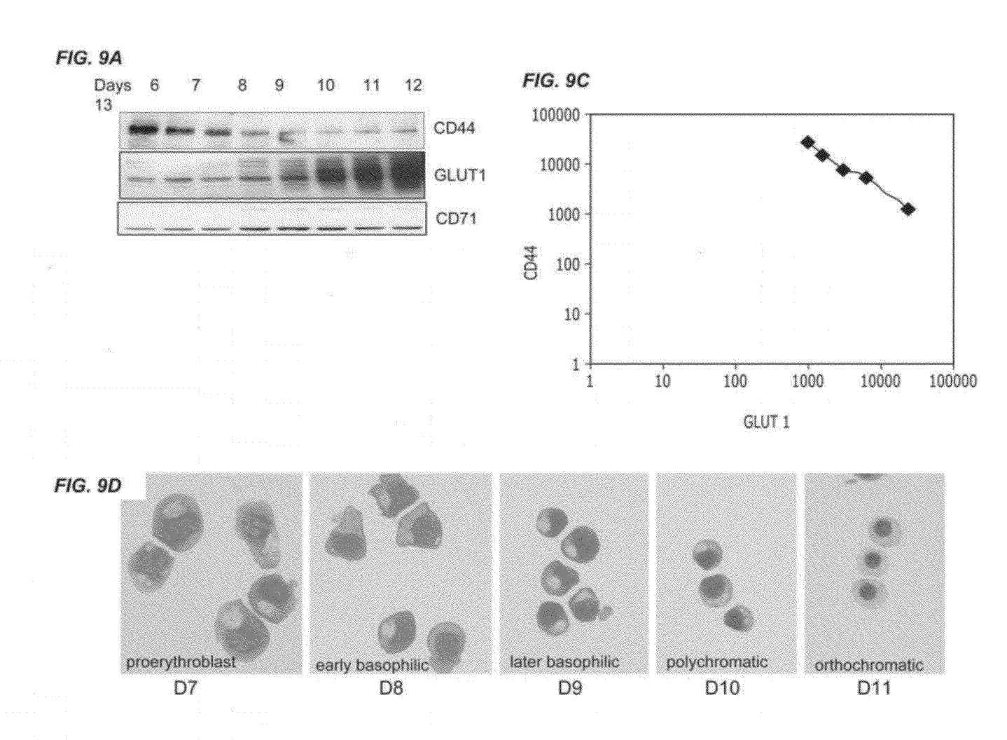

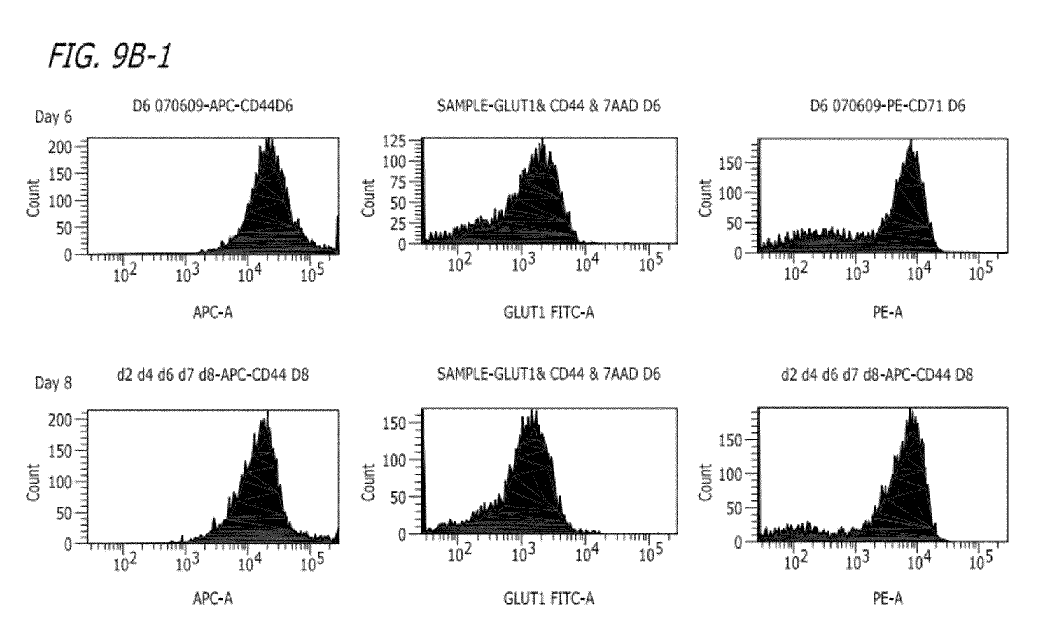

Two distinct erythroid progenitors have been functionally defined in colony assays, namely, the early-stage burst forming unit-erythroid (BFU-E), and the later-stage colony forming unit-erythroid (CFU-E) progenitor. The earliest morphologically recognizable erythroblast in hematopoietic tissues is the proerythroblast, which undergoes three to four mitoses to produce reticulocytes. Morphologically distinct populations of erythroblasts are produced by each successive mitosis, beginning with proerythroblasts and followed by basophilic, polychromatic and orthochromatic erythroblasts. Based on the changes in expression levels of CD44, GPA and cell size, a method was developed to isolate populations of erythroblasts at each stage of development, in a more homogenous state than previously achieved, dependent on the expression levels of the transferrin receptor, CD71. In this study, the ratio of proerythroblast:basophilic:polychromatic:orthochromatic follows the 1:2:4:8 manner, demonstrating the doubling of cells upon each mitosis. Progression of erythropoiesis is normal in 4.1R-knockout mice (a hemolytic anemia model), however in thalasemia mice the progression from proerythroblasts to basophilic erythroblasts is altered (Table 2). Thus the disclosed method now enables the quantification of in vivo erythropoiesis of mouse bone marrow and to define stage-specific defects in erythroid maturation in inherited red cell disorders. Flow cytometry of mouse bone marrow cells is depicted in FIG. 8A-8I.

TABLE 2
Quantitative Analysis of Erythropoiesis of Mouse Bone Marrow

| | WT | 4.1R KO | Thalassemia |
|---|---|---|---|
| proerythroblast | 5.4 ± 0.6 | 5.5 ± 0.4 | 2.0 ± 0.8 |
| basophilic | 13.0 ± 0.5 | 12.8 ± 0.3 | 11.7 ± 4.5 |
| polychromatic | 28.0 ± 2.0 | 27.8 ± 0.5 | 29.4 ± 1.9 |
| orthochromatic | 53.0 ± 1.7 | 53.9 ± 0.4 | 54.2 ± 1.6 |

Methods

Harvest cells. Bone marrow cells were harvested from tibia and femur of 3-month-old mice.

CD45 positive cells depletion. The cell number was determined and then the cells were centrifuged at 300×g for 10 min. The supernatant was removed and the cell pellet was resuspended in 90 μL of buffer per 10⁷ total cells. Next, 10 μL of CD45 MicroBeads were added per 10⁷ total cells and the cell suspension was mixed well and incubated for 15 min at 4-8° C. The cells were then washed by adding 1-2 mL of buffer per 10⁷ cells and centrifuged at 300×g for 10 min. The supernatant was removed and the cell pellet was resuspended at 10⁸ cells in 500 μL of buffer. This cell population was then used for magnetic separation.

Magnetic separation with MS or LS columns. Magnetic separation was conducted according to the column's manufacturer's instructions. The cells which pass through the columns were collected in a clear 15 mL tube, washed twice in 1 mL of buffer and the total effluent was collected.

Staining. 2×10⁶ cells were resuspended in 80 μL DPBS/0.5% BSA and the cells were blocked with rat anti-mouse CD16/CD32 (1 μg/10⁶ cells) for 15 min. Samples were subsequently stained with FITC rat anti-mouse TER119 (1 μg/10⁶ cells), APC rat anti-mouse CD44 (0.5 μg/10⁶ cells) and APC-Cy™ 7 rat anti-mouse CD11b (0.2 μg/10⁶ cells) on ice for 20-30 min in the dark. Cells were washed twice with 700 μL DPBS/0.5% BSA. Finally, the cells were resuspended in 100 μL DPBS/0.5% BSA and stained with the viability marker 7-AAD (0.25 μg/10⁶ cells) on ice for 10 min in the dark. The cells were then suspended in 0.4 mL of PBS/0.5% BSA and analyzed within 1 hr following staining by flow cytometry. Unstained cells were used as a negative control.

### Example 3

### Analysis of In Vitro Human Erythropoiesis

Erythropoiesis is the process by which nucleated erythroid progenitors proliferate and differentiate to generate, every second, millions of non-nucleated red cells with their unique discoid shape and membrane material properties. The time-course appearance of individual membrane protein components during murine erythropoiesis was studied and distinct changes of individual proteins were found during terminal erythroid differentiation, particularly a progressive and dramatic decrease of CD44 from proerythroblast to reticulocyte. These findings have allowed the development of a new strategy for quantifying the in vivo erythropoiesis and defining stage-specific defects in erythroid maturation in mouse. To examine whether the similar strategy can be applied to study human erythropoiesis, the expression of various red cell membrane proteins was examined during terminal differentiation of human erythroid progenitors using a human unilineage erythroid culture system.

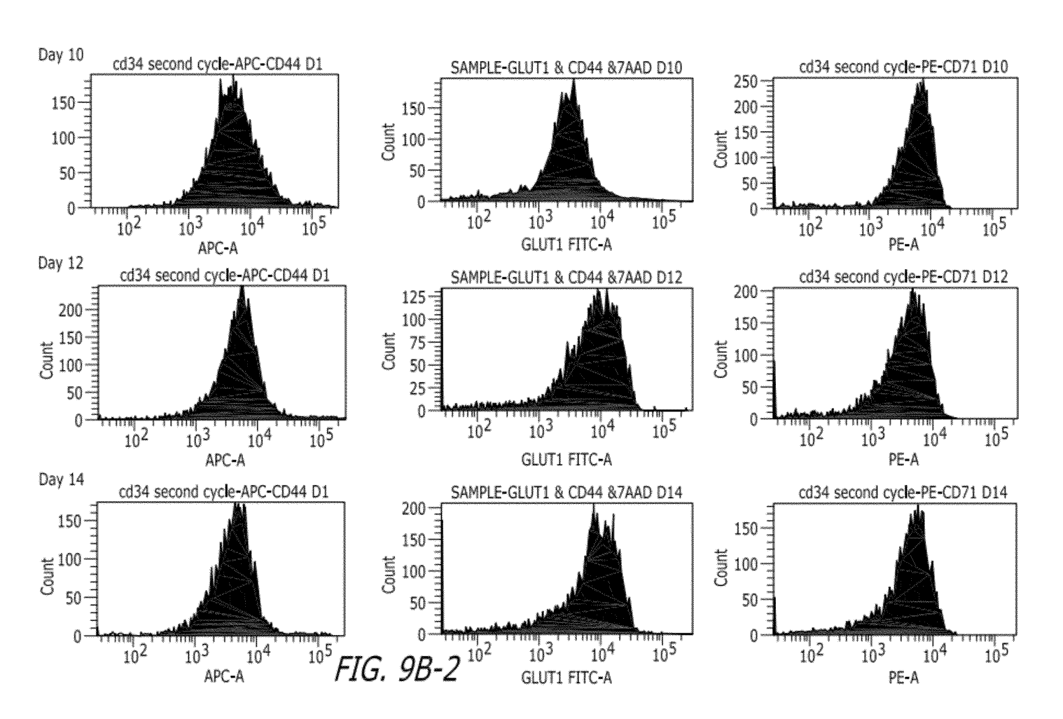

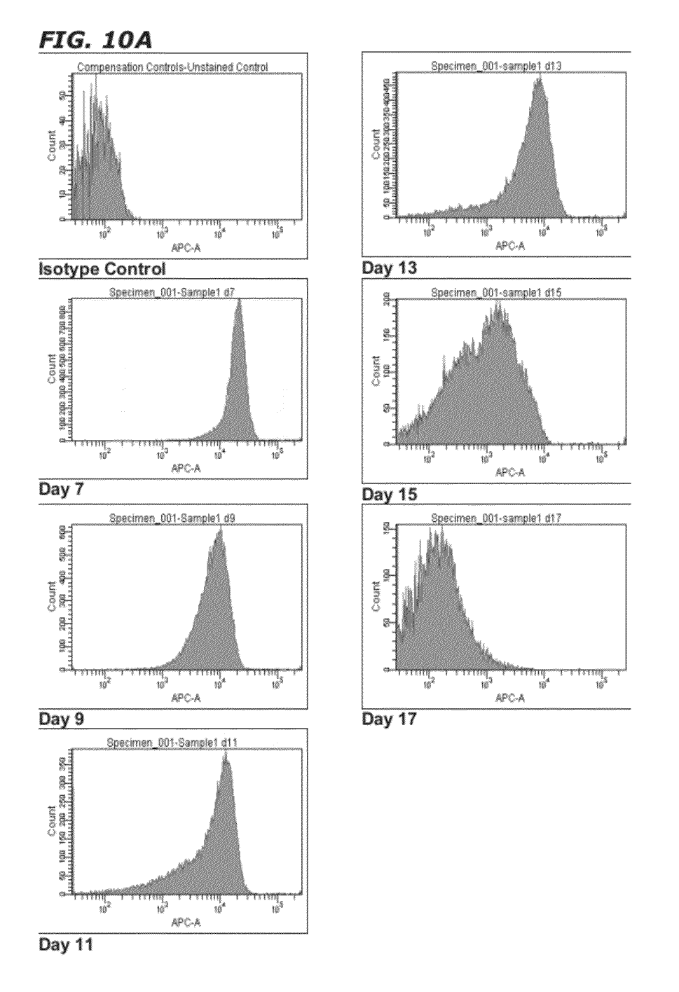

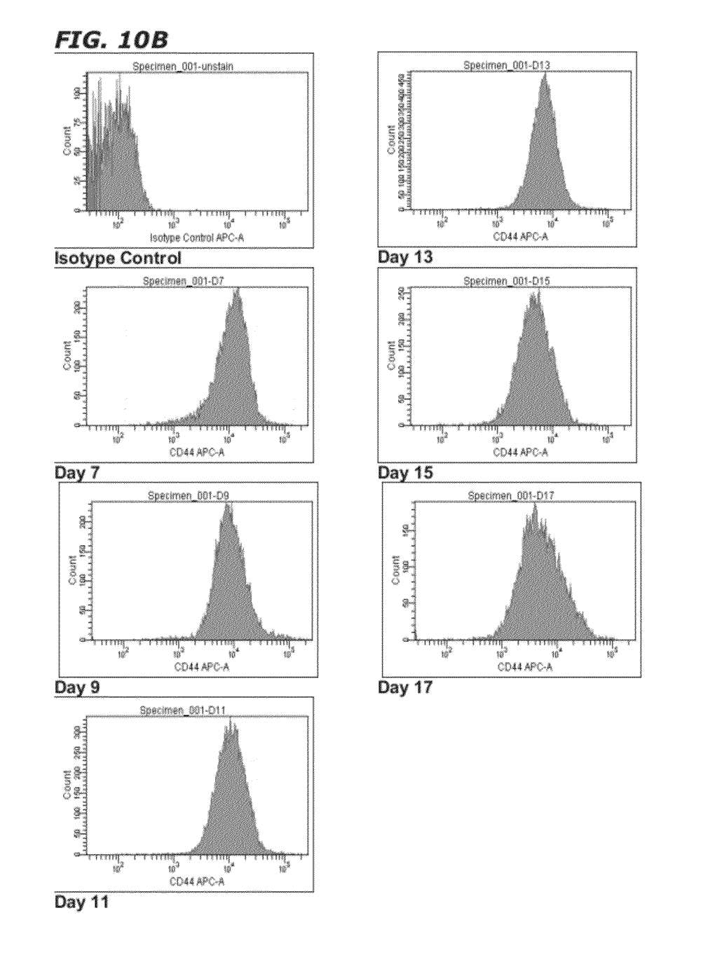

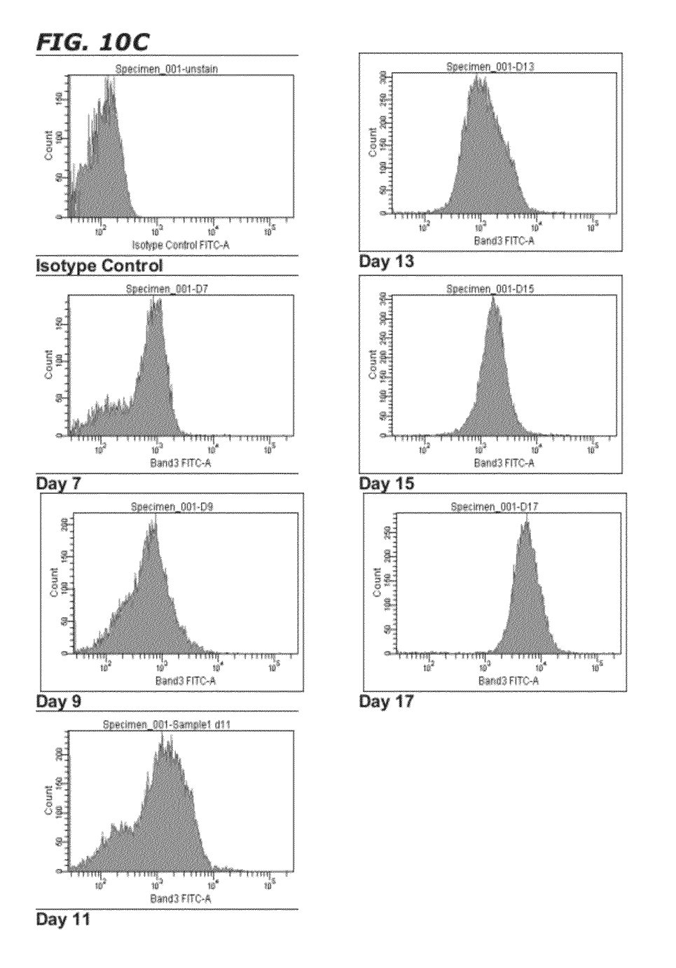

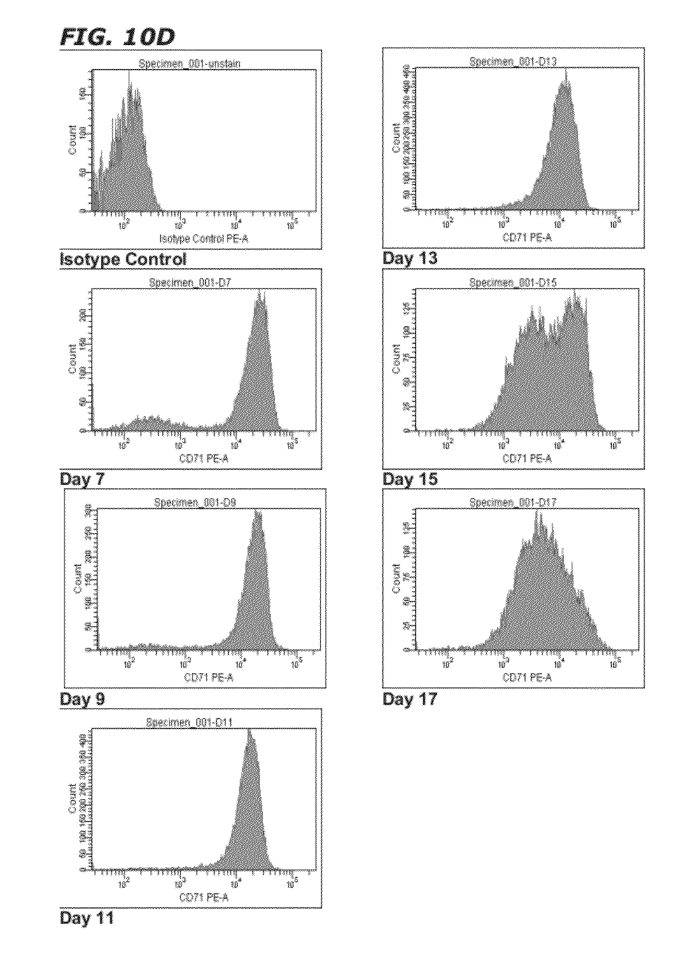

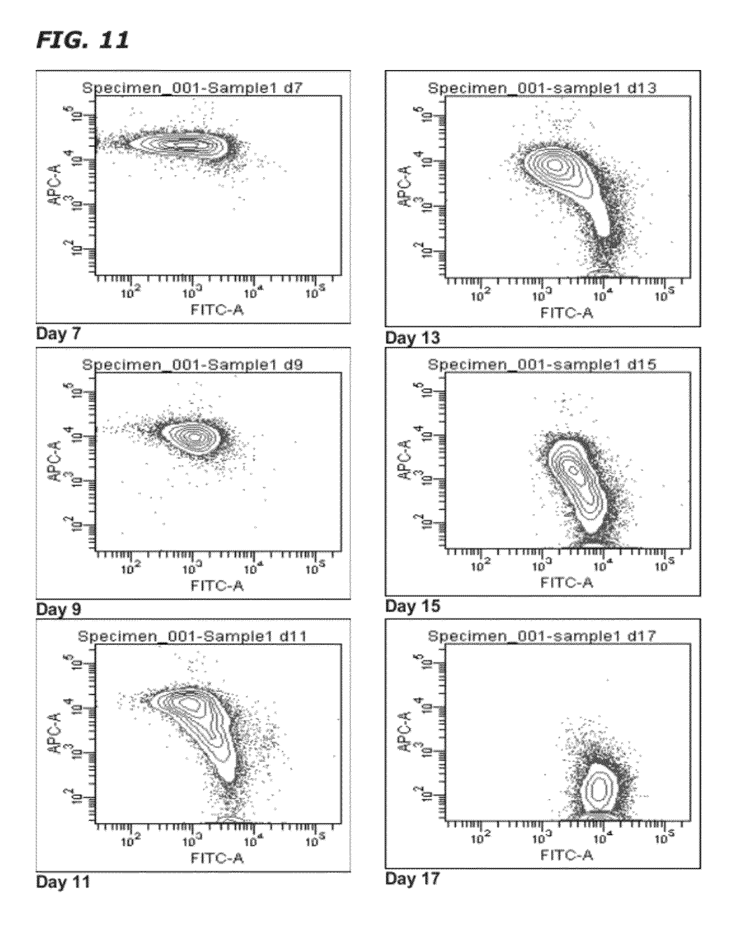

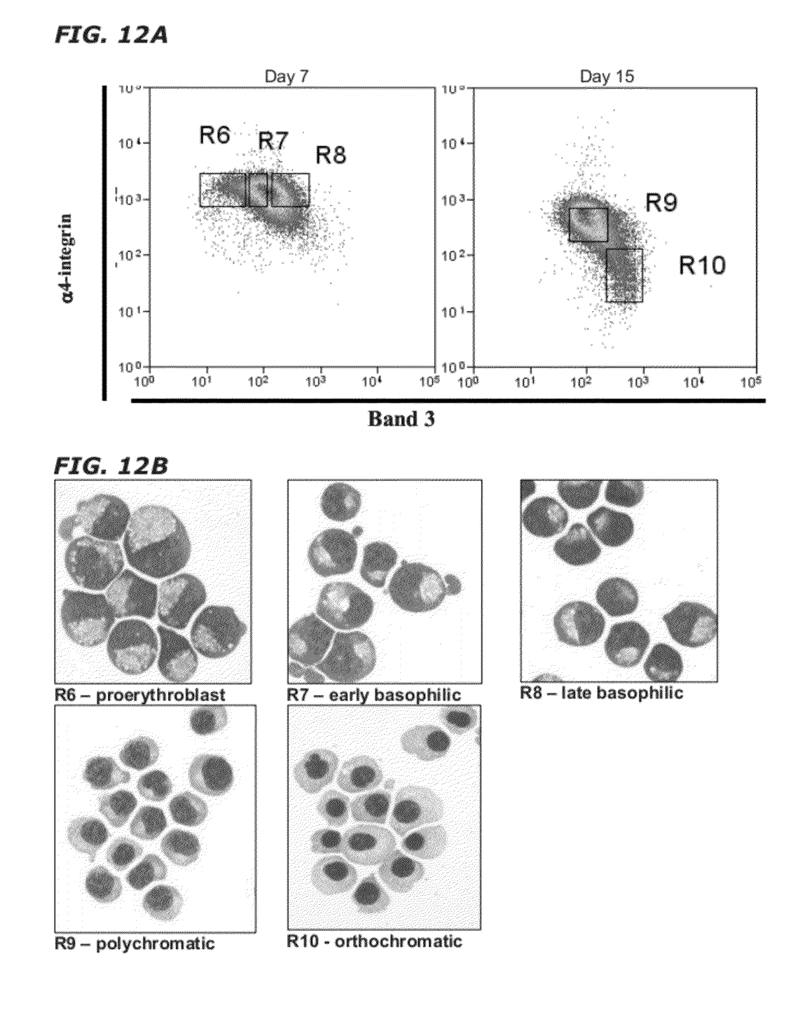

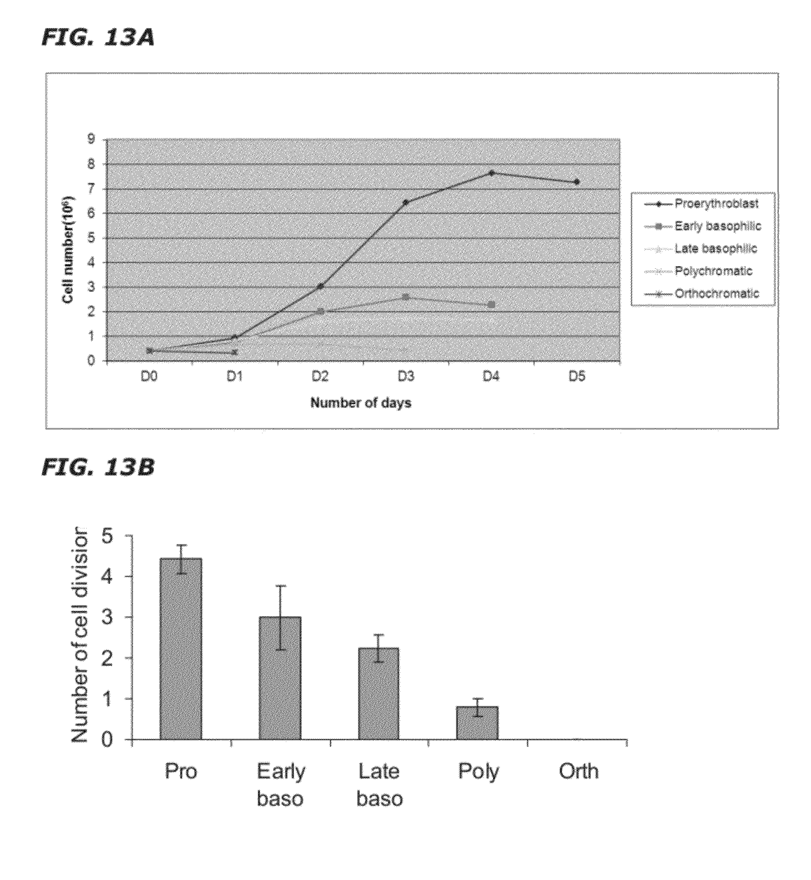

Human erythroblasts were stained with RhAg, Kell, GPC, α4 integrin, GPA, band 3, CD71, CD36, CD59, β1 integrin, and α5 integrin and analyzed by flow cytometry. Surprisingly, the human erythroblasts exhibited different staining phenotypes than mouse cells. Particularly, α4 integrin and band 3 were increased, while CD44 and CD71 did not exhibit obvious changes (FIG. 10). Using α4 integrin and band 3 as surface markers, it was possible to isolate distinct stages of erythroblasts from in vitro cultured erythroblasts by FACS (Fluorescence Activated Cell Sorting) (FIGS. 11 and 12). These findings strongly suggest that α4 integrin in conjunction with band 3 can be used to distinguish erythroblasts at successive developmental stages during human erythropoiesis, which offers a means of defining stage-specific defects in erythroid maturation in inherited and acquired red cell disorders and in bone marrow failure syndromes.

Methods

Isolation of mononuclear cells using Ficoll-Paque. Core blood was diluted 2-4 volumes with buffer and the mononuclear cells were separated with Ficoll isolation by density gradient centrifugation. The interphase layer cells were transferred to a new 50 mL conical tube, washed to 4 times with buffer and resuspend in appropriate amount of buffer prior to magnetic separation.

CD34+ Magnetic Labeling. The isolated cells were spun down at 200×g for 3-5 min, 4° C. For every 1×10⁸ of cells, the cells were resuspended in 300 μL buffer and 100 μL of FcR Blocking Reagent and 100 μL of CD34 microbeads were added. The cells were mixed well and incubated at 4° C. for 30 min. The cells were then washed in 2-5 mL of buffer and resuspended in 1-2 mL of buffer.

Magnetic separation with LS columns. Magnetic separation was conducted according to the column's manufacturer's instructions. Briefly, the column was prepared by rinsing with appropriate amount of buffer and the cell suspension was applied to the column. The column was washed with 3×3 mL of buffer. The column was then removed the separator and placed in a suitable collection tube and the magnetically labeled cells were flushed from the column by firmly pushing the plunger into the column. The cells were counted and spun down at 200×g for 3-5 min at 4° C.

Cell culture. Cells were cultured by two steps protocol. In the first step (day 0-day 6), 10⁵/mL CD34+ cells were cultured (day 0) in Serum-Free Expansion Medium (SFEM, Stem Cell Technologies) supplemented with 10% fetal bovine serum (FBS), 50 ng/mL Stem Cell Factor (SCF), 10 ng/mL IL-3, 1 U/mL erythropoietin (EPO), α-thioglycerol (0.6 μL/mL medium after 1:00 dilution) and penicillin-streptomycin 100×. After 4 days in culture, the cells were diluted to 10⁵/mL using fresh medium and continue to culture three days. In the second step (day 7-day 13), cells were cultured at 10⁵/mL in SFEM medium supplemented with 30% FBS, with the same concentration of EPO, α-thioglycerol and penicillin-streptomycin.

Cell staining and sorting. In one example, 5×10⁶ cells were resuspended in 400 μL DPBS/0.5% BSA. Samples were stained with APC rat anti-mouse CD44 (0.5 μg/10⁶ cells) on ice for 20-30 min in the dark. Cells were washed twice with 700 μL DPBS/0.5% BSA. Then, cells were resuspended in 400 μL DPBS/0.5% BSA and stained with the GFP-GLUT1 antibody at 37° C. for 30 min in the dark. After washing, the cells were resuspended in 400 mL DPBS/0.5% BSA and stained with the viability marker 7-AAD (0.25 μg/10⁶ cells) on ice for 10 min in the dark. Unstained cells were used as a negative control. Single color stain sample were used as compensation. The sample then analyzed within 1 hr following staining by flow cytometry. The same staining protocol was used for the following additional markers: RhAg, Kell, GPC, α4 integrin, GPA, band 3, CD71, CD36, CD59, β1 integrin, and α5 integrin.

### Example 4

### Mitotic Ability of Purified Human Erythroblasts

In order to determine the mitotic ability of the purified human erythroblasts, the cells were cultured at a concentration of 10⁵/mL in SFEM medium supplemented with 30% FBS, 1 U/mL EPO, α-thioglycerol (0.6 μL/mL medium after 1:00 dilution) and penicillin-streptomycin 100×. Ten microliters of cell suspension were removed daily and the cell number determined.

Proerythroblasts exhibited the highest mitotic ability, followed by early basophilic, late basophilic, polychromatic, and orthochromatic erythroblasts.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for isolating erythroid cells at different developmental stages comprising identifying the desired erythroid cells in a source of human erythroid cells, wherein the isolated erythroid cells express a level of α4 integrin and band 3 is indicative of a developmental stage of the erythroid cells.

2. The method according to claim 1, wherein α4 integrin expression decreases and band 3 expression increases as differentiation of the erythroid cells progresses.

3. The method according to claim 1, further comprising quantifying the number of erythroid cells in the developmental stage.

4. The method of claim 1, wherein the erythroid cells are from a source selected from peripheral blood, bone marrow, cord blood, apheresis cells, and placenta.

5. A method for identifying the erythroid cell maturation stage in an RBC disorder comprising determining the expression of α4 integrin and band 3 on erythroid cells from an individual having a disorder of erythropoiesis.

6. The method according to claim 5, wherein the RBC disorder is selected from a thalassemia, an RBC maturation disorder, and a bone marrow failure syndrome.

7. The method according to claim 6, wherein the RBC maturation disorder is a myelodysplastic syndrome.

8. The method according to claim 6, wherein the thalassemia is Cooley's anemia.

9. The method according to claim 5, wherein the number of erythroid cells of a particular maturation stage in quantified in the RCS disorder.

10. A method for monitoring ex vivo proliferation and differentiation of stem cells into hematopoietic precursors, erythroid cells, and mature red blood cells comprising determining the expression of α4 integrin and band 3 on the cells, wherein the level of expression of α4 integrin and band 3 is indicative of a developmental stage of the erythroid cells.

11. The method of claim 10, wherein the hematopoietic precursor is a red blood cell precursor.

12. The method of claim 10, further comprising quantifying the number of hematopoietic precursors, RBC precursors, or mature RBCs in an RBC maturation or differentiation stage.

13. The method of claim 10, wherein the erythroid cells are from a source selected from peripheral blood, bone marrow, cord blood, apheresis, stem cells, and placenta.

14. The method of claim 13, wherein the stem cells are embryonic stem cells.
